(19) United States
(12) Patent Application Publication
(10) Pub. No.: US 2023/0289741 A1
(43) Pub. Date: Sep. 14, 2023

(54) TECHNIQUES FOR FACILITATING VETERINARY SERVICES

(71) Applicant: Better Vet LLC, Hingham, MA (US)

(72) Inventors: Matt HARRIS, Quincy, MA (US); Michael BORSARI, Fairfield, CT (US); Bruce HERZFELDER, Westwood, MA (US)

(21) Appl. No.: 18/179,571

(22) Filed: Mar. 7, 2023

Related U.S. Application Data
(60) Provisional application No. 63/317,813, filed on Mar. 8, 2022.

(51) Int. Cl. G06Q 10/1093; G06Q 10/0631; G01C 21/34; G01C 21/36

(52) U.S. Cl. CPC G06Q 10/1095; G06Q 10/06311; G01C 21/3415; G01C 21/3446; G01C 21/3617

(57) ABSTRACT

Various embodiments are generally directed to techniques for facilitating veterinary services, such as with a schedule administrator, for instance. Many embodiments include a schedule administrator for generating schedule data based on planning data. In many such embodiments, the schedule data may be dynamically updated based on updated, or additional, planning data. In various embodiments, the schedule data may include a schedule of appointments for veterinary services. In some embodiments, the schedule data may include routes for veterinary service providers. In several embodiments, the schedule administrator may utilize one or more artificial intelligence algorithms to generate one or more portions of the schedule data, such as the schedule of appointments and/or the routes for veterinary service providers. In many embodiments, the artificial intelligence algorithms may be trained on historical data comprising a plurality of historical appointments.

### CROSS-REFERENCE TO RELATED APPLICATIONS

The present application hereby claims priority to, and incorporates by reference the entirety of the disclosure of, U.S. Provisional patent application 63/317,813, entitled “TECHNIQUES FOR FACILITATING VETERINARY SERVICES”, filed on Mar. 8, 2022.

### BACKGROUND

Modern veterinary practice requires that animals be taken to a clinic or hospital, where the veterinary service is carried out. However, clinic and hospital environments often cause stress and anxiety to the animals. Where the animal is sick or their behavior indicates some possible underlying condition, a stressful environment may not provide the best location for performing a checkup or administering veterinary services. For example, performing routine checkups in such a stressful environment may skew indicators used to diagnose illness or track the general health and well-being of the animal. Further still, continued monitoring and subsequent checkups for animals may not be carried out due to the inconvenience of repeatedly taking the animal to the clinic and due to the desire to avoid repeated stress of the animal. Where the animal is recovering from an illness or injury, the desire to avoid repeatedly stressing the animal may lead to less-than-optimal care from a veterinarian.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates an exemplary operating environment for a schedule administrator according to one or more embodiments disclosed hereby.

FIG. 2A illustrates various aspects of appointment data according to one or more embodiments disclosed hereby.

FIG. 2B illustrates various aspects of client data according to one or more embodiments disclosed hereby.

FIG. 2C illustrates various aspects of pet data according to one or more embodiments disclosed hereby.

FIG. 2D illustrates various aspects of provider data according to one or more embodiments disclosed hereby.

FIG. 2E illustrates various aspects of zone data according to one or more embodiments disclosed hereby.

FIG. 2F illustrates various aspects of historical data according to one or more embodiments disclosed hereby.

FIG. 3A illustrates various aspects of a schedule administrator according to one or more embodiments disclosed hereby.

FIG. 3B illustrates various aspects of schedule data according to one or more embodiments disclosed hereby.

FIG. 4A illustrates an exemplary process flow for an availability manager according to one or more embodiments disclosed hereby.

FIG. 4B illustrates an exemplary process flow for a duration estimator according to one or more embodiments disclosed hereby.

FIG. 4C illustrates an exemplary process flow for a dynamic route planner according to one or more embodiments disclosed hereby.

FIG. 5 illustrates a pet owner computing device according to one or more embodiments disclosed hereby.

FIG. 6 illustrates a vet service provider computing device according to one or more embodiments disclosed hereby.

FIGS. 7A and 7B illustrate networked systems according to one or more embodiments disclosed hereby.

FIG. 8 illustrates an exemplary process flow according to one or more embodiments disclosed hereby.

FIG. 9 illustrates an exemplary logic flow according to one or more embodiments disclosed hereby.

FIGS. 10A and 10B illustrate an exemplary logic flow according to one or more embodiments disclosed hereby.

FIG. 11 illustrates an exemplary logic flow according to one or more embodiments disclosed hereby.

FIG. 12 illustrates a computer-readable storage medium according to one or more embodiments disclosed hereby.

FIG. 13 illustrates a diagrammatic representation of a machine 1200 in the form of a computer system within which a set of instructions may be executed for causing the machine to perform any one or more of the methodologies discussed herein, according to one or more embodiments disclosed hereby.

### DETAILED DESCRIPTION

Various embodiments are generally directed to techniques for facilitating veterinary services, such as with a schedule administrator, for instance. Many embodiments include a schedule administrator for generating schedule data based on planning data. In many such embodiments, the schedule data may be dynamically updated based on updated, or additional, planning data. In various embodiments, the schedule data may include a schedule of appointments for veterinary services. In some embodiments, the schedule data may include routes for veterinary service providers. In several embodiments, the schedule administrator may utilize one or more artificial intelligence algorithms to generate one or more portions of the schedule data, such as the schedule of appointments and/or the routes for veterinary service providers. In many embodiments, the artificial intelligence algorithms may be trained on historical data comprising a plurality of historical appointments. These and other embodiments are described and claimed.

Some challenges facing facilitating veterinary services include rigid and inefficient techniques for scheduling appointments. For example, high priority appointments (e.g., emergency appointments) may be scheduled without regard to previously scheduled appointments (e.g., routine appointments), resulting in unannounced delays for the previously scheduled appointments and poor client experiences. In another example, appointments may not be available at customizable locations, such as a client residence, limiting the ability to provide veterinary services in an adaptable manner. Accordingly, clients may be required to go to a veterinary clinic to obtain services for an animal. Alternatively, appointments at customized locations may require additional fees or only be available at limited times. Additionally, allowing customizable locations for veterinary services in an efficient and adaptable manner introduces significant logistical challenges. For example, generating and changing schedules for appointments must account for travel times as well as constantly changing locations of the service providers. Further, generating efficient routes for service providers to visit a variety of locations is an exceedingly difficult problem that is only complicated by last minutes changes. In another example, different locations provide different and challenging environments for providing the veterinary services. These and other factors may result in veterinary services with limited adaptability, deficient quality, poor client experiences, and reduce reliability. Such limitations can drastically reduce the accessibility, usability, and scalability of veterinary services, contributing to excessive costs, lost revenues, and limited availability.

Various embodiments described hereby include a computerized schedule administrator that is able to facilitate veterinary services efficiently and effectively in a scalable and dynamic manner. In many embodiments, the schedule administrator may utilize artificial intelligence to schedule appointments and/or route service providers automatically, or with little to no human intervention. Several embodiments may estimate service durations (i.e., service times) in an accurate and reliable manner, such as based on historical data on appointments. The estimated service durations may in turn be used to generate accurate, reliable, and achievable schedules and/or service routes in a dynamic manner. In many embodiments, the schedule administrator may track services with a variety of metrics to continually improve the provision of veterinary services, such as by generating detailed historical data for training artificial intelligence algorithms for estimating service durations or routing service providers. In many embodiments, the schedule administrator can facilitate and adapt to schedule changes in an efficient manner. For example, emergency appointments may be seamlessly integrated into schedules and routes without wasting time or resources. In another example, schedules and routes may be automatically updated in response to cancellations. Additionally, the schedule administrator may handle client and/or service provider communications, such as by updating clients and service providers to schedule and route changes.

In these and other ways, components/techniques described hereby may achieve significant improvement in the computerized facilitation of veterinary services, resulting in several technical effects and advantages over conventional computer technology, including increased capabilities and improved performance. In various embodiments, one or more of the aspects, techniques, and/or components described hereby may be implemented in a practical application via one or more computing devices, and thereby provide additional and useful functionality to the one or more computing devices, resulting in more capable, better functioning, and improved computing devices. For example, a practical application may include a schedule administrator that utilizes historical appointment data for one or more of estimating appointment durations, scheduling appointments, and routing service providers with artificial intelligence algorithms. In yet another example, the practical application may include collecting historical appointment data for training an artificial intelligence algorithm to perform one or more of estimating service durations, scheduling services, and routing service providers. In another example, the practical application may include utilizing estimated appointment durations and/or appointment locations to schedule appointments and/or generate routes for services providers. In yet another example, the practical application may include receiving updated or additional information and automatically rescheduling appointments, rerouting service providers, and/or notifying interested parties in response. Further, one or more of the aspects, techniques, and/or components described hereby may be utilized to improve the technical fields of logistics, service administration, veterinary practice, schedule generation, route planning, and artificial intelligence.

In several embodiments, components described hereby may provide specific and particular manners to enable development, evaluation, management, and optimization of artificial intelligence algorithms. In many embodiments, one or more of the components described hereby may be implemented as a set of rules that improve computer-related technology by allowing a function not previously performable by a computer that enables an improved technological result to be achieved. For example, the function allowed may include one or more of the specific and particular techniques disclosed hereby. For example, the specific and particular techniques many include providing one or more of historical appointment data, client data, animal data, provider data, and zone data to an artificial intelligence algorithm to estimate a veterinary service appointment duration, generate a schedule of veterinary service appointments, and/or generate a route for a provider of veterinary services. In another example, the specific and particular techniques many include training an artificial intelligence algorithm based on one or more of historical appointment data, client data, animal data, provider data, and zone data. In yet another example, the specific and particular techniques many include automatically collecting one or more of appointment data, client data, animal data, provider data, and zone data to produce historical data for training artificial intelligence algorithms.

With general reference to notations and nomenclature used hereby, one or more portions of the detailed description which follows may be presented in terms of program procedures executed on a computer or network of computers. These procedural descriptions and representations are used by those skilled in the art to effectively convey the substances of their work to others skilled in the art. A procedure is here, and generally, conceived to be a self-consistent sequence of operations leading to a desired result. These operations are those requiring physical manipulations of physical quantities. Usually, though not necessarily, these quantities take the form of electrical, magnetic, or optical signals capable of being stored, transferred, combined, compared, and otherwise manipulated. It proves convenient at times, principally for reasons of common usage, to refer to these signals as bits, values, elements, symbols, characters, terms, numbers, or the like. It should be noted, however, that all of these and similar terms are to be associated with the appropriate physical quantities and are merely convenient labels applied to those quantities.

Further, these manipulations are often referred to in terms, such as adding or comparing, which are commonly associated with mental operations performed by a human operator. However, no such capability of a human operator is necessary, or desirable in many cases, in any of the operations described hereby that form part of one or more embodiments. Rather, these operations are machine operations. Useful machines for performing operations of various embodiments include general purpose digital computers as selectively activated or configured by a computer program stored within that is written in accordance with the teachings hereby, and/or include apparatus specially constructed for the required purpose. Various embodiments also relate to apparatus or systems for performing these operations. These apparatuses may be specially constructed for the required purpose or may include a general-purpose computer. The required structure for a variety of these machines will be apparent from the description given.

Reference is now made to the drawings, whereby like reference numerals are used to refer to like elements throughout. In the following description, for purpose of explanation, numerous specific details are set forth in order to provide a thorough understanding thereof. It may be evident, however, that the novel embodiments can be practiced without these specific details. In other instances, well known structures and devices are shown in block diagram form to facilitate a description thereof. The intention is to cover all modification, equivalents, and alternatives within the scope of the claims.

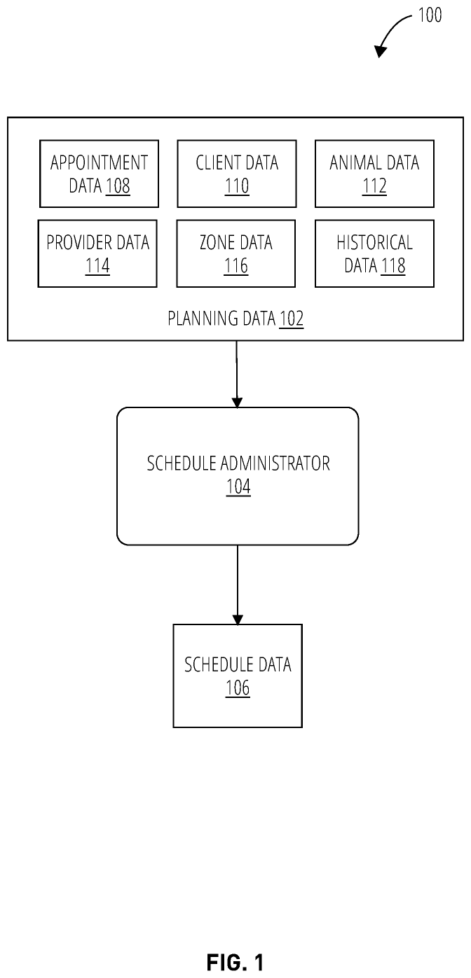

FIG. 1 illustrates an exemplary operating environment 100 for a schedule administrator 104 according to one or more embodiments described hereby. Operating environment 100 may include the schedule administrator 104 in conjunction with planning data 102 and schedule data 106. In various embodiments, the schedule administrator 104 may utilize planning data 102 to generate schedule data 106 for facilitating veterinary services. In several embodiments, schedule data 106 may include one or more of a schedule of appointments for veterinary services and routes for providers of the veterinary services. In the illustrated embodiment, planning data 102 includes appointment data 108, client data 110, animal data 112, provider data 114, zone data 116, and historical data 118. In many embodiments, the schedule administrator 104 may collect and/or generate one or more portions of the planning data 102. In some embodiments, FIG. 1 may include one or more components that are the same or similar to one or more other components of the present disclosure. Further, one or more components of the present disclosure, or aspects thereof, may be incorporated into, or excluded from, the embodiment of FIG. 1 without departing from the scope of this disclosure.

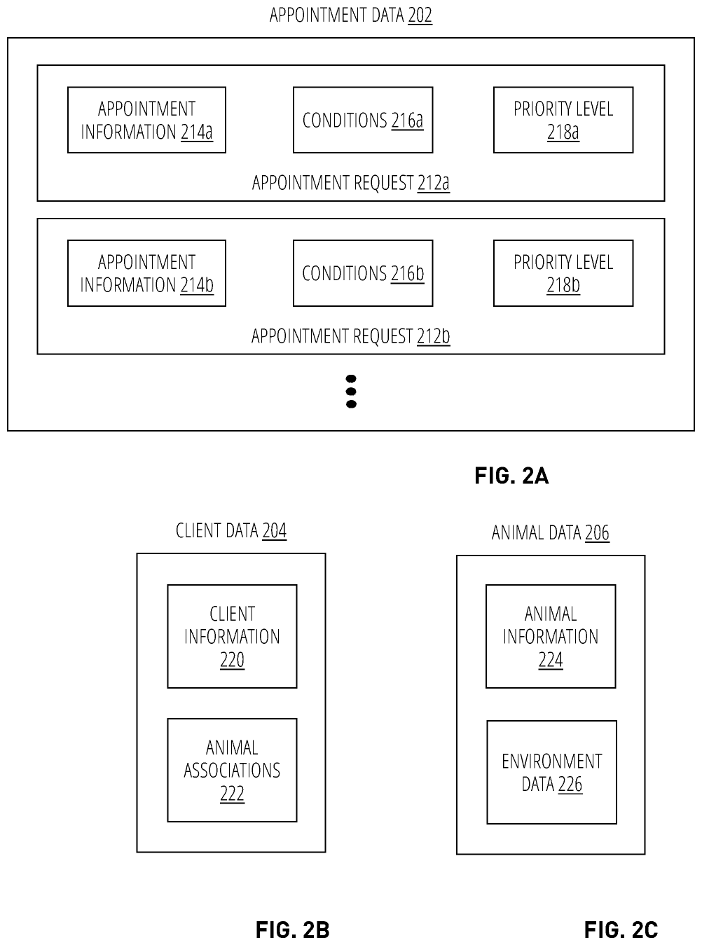

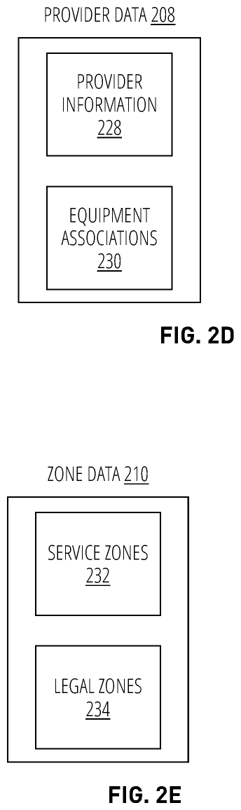

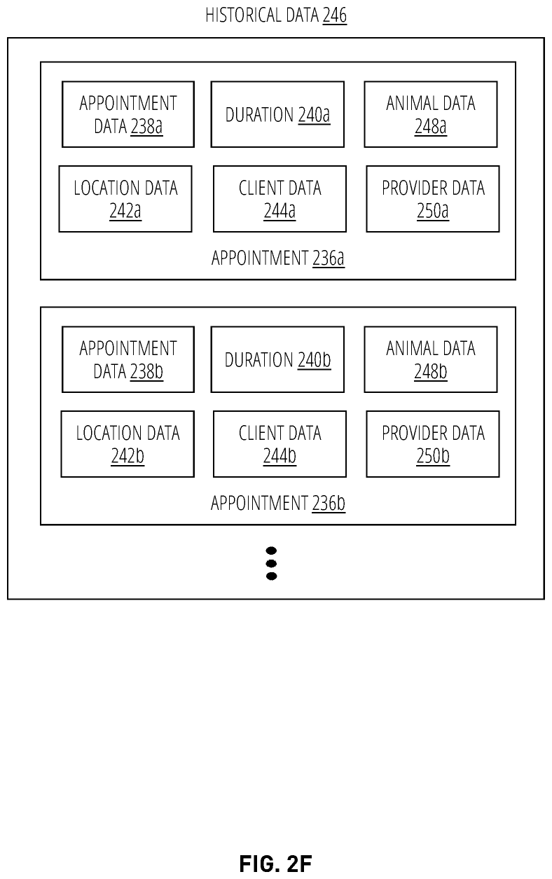

FIGS. 2A-2F illustrate various aspects of planning data according to one or more embodiments described hereby. More specifically, FIG. 2A illustrates exemplary appointment data 202; FIG. 2B illustrates exemplary client data 204; FIG. 2C illustrates exemplary animal data 206; FIG. 2D illustrates exemplary provider data 208; FIG. 2E illustrates exemplary zone data 210; and FIG. 2F illustrates exemplary historical data 246. In various embodiments, various portions of the planning data may be utilized, collected, and/or generated to realize one or more embodiments described hereby. In some embodiments, FIGS. 2A-2F may include one or more components that are the same or similar to one or more other components of the present disclosure. Further, one or more components of the present disclosure, or aspects thereof, may be incorporated into, or excluded from, the embodiments of FIGS. 2A-2F without departing from the scope of this disclosure.

Referring to FIG. 2A, appointment data 202 is shown. In the illustrated embodiment, appointment data 202 includes appointment request 212a and appointment request 212b. However, it will be appreciated that appointment data 202 may include any number of appointment requests, such as a number based on the number of appointment requests received from clients. Appointment request 212a includes appointment information 214a, conditions 216a, and priority level 218a and appointment request 212b includes appointment information 214b, conditions 216b, and priority level 218b. In some embodiments, the appointment requests 212 may be received via a user interface. In some such embodiments, the user interface may be provided, or generated, by the schedule administrator.

Appointment information (e.g., 214a, 214b) may include data relevant to the requested appointment that is received as part of the appointment request. For example, appointment information may include one or more of a veterinary service type, an animal type, a location type, an address, a number of animals, an associated client, and the like. A veterinary service type, for example, may include a routine check-up, a delivery, a vaccination, a diagnosis, a euthanasia, a physical, etcetera. Location type may include apartments, single family homes, farms, urban, rural, suburban, and the like. Conditions (e.g., 216a, 216b) may include various preferences, requests, or requirements for the requested appointment. For example, conditions may include preferred, requested, and/or required service providers, dates, times, required skills of the provider, required equipment, and the like. In another example, conditions may include a class of service, such as remote, virtual, in-home, or hybrid virtual-in-home veterinary service. Priority levels may include a priority level associated with the request. For example, a first priority level may be associated with emergencies and another priority level may be associated with routine visits. In various embodiments, appointment requests may include information relevant to estimating a veterinary service appointment duration, generating a schedule of veterinary service appointments, and/or generating a route for a provider of veterinary services. For example, appointment requests may indicate whether the requested service is to be performed as a remote, virtual, in-home, or hybrid virtual-in-home veterinary service.

In some embodiments, the priority level may be included as a condition. In various embodiments, the priority level may be assigned by the schedule administrator. In some embodiments, the priority level may be selected, or requested, by the requestor. In some such embodiments, the priority level may be reassigned by the schedule administrator. In one embodiment, priority levels may be utilized to determine the specificity with which an appointment is scheduled. For example, low priority appointments may be scheduled for a time window while high priority appointments are scheduled for a specific time. In some embodiments, different appointment requests may include different amounts or types of information. For example, if the appointment request is associated with a registered, or otherwise known, animal it may include less information than if the appointment request is associated with an unregistered, or otherwise unknown, animal.

Referring to FIG. 2B, client data 204 is shown. In the illustrated embodiment, client data 204 includes client information 220 and animal associations 222. Client information 220 may include data relevant to a client that is utilized or collected for various aspects of providing veterinary services. For example, client information may include personal information, billing data, insurance information, location data, preferences, habits, tendencies, and the like. For example, preferences may include preferred service providers, preferred times, preferred dates, and the like. Client data 204 may be generated for each client in the system. Accordingly, client data 110 in planning data 102 may include a plurality of collections of client data 204. In some embodiments, a client may include multiple individuals, such as a family or a business.

In various embodiments, client data 204 may include information relevant to estimating a veterinary service appointment duration, generating a schedule of veterinary service appointments, and/or generating a route for a provider of veterinary services. For example, tendencies may include that a client typically has numerous questions. In another example, habits may include that a client typically requests additional services once an appointment has begun or whether a client is typically prepared or unprepared for an appointment (e.g., has animal prepared). In yet another example, location data may include a location type and/or geographical information associated with the client. In some embodiments, a service time for an appointment may include transit time between locations. In other embodiments, transit times may be estimated separate from service times and added in. Animal associations 222 may include one or more animals that are associated with the client. In some embodiments, animal associations 222 may include, or identify, one or more collections of animal data. For example, animal associations 222 may refer to a collection of animal data (e.g., animal data 206) for each animal the client is associated with. In one embodiment, animal associations 222 may relate the client associated with client data 204 to each animal they have had veterinary services performed for.

Referring to FIG. 2C, animal data 206 is shown. In the illustrated embodiment, animal data 206 includes animal information 224 and environment data 226. Animal information 224 may include data relevant to an animal that is utilized or collected for various aspects of providing veterinary services. For example, animal information may include medical history, billing data, insurance information, location data, client associations, animal associations, preferences, habits, tendencies, temperaments, and the like. In some embodiments, animal information may include data indicative of at least one historical interaction with the animal. For example, preferences may include preferred service providers. In another example, preferences may include belly rubs or that the animal likes to be with a companion animal. In yet another example, temperaments may include that an animal is a biter. Animal data 206 may be generated for each animal in the system. Accordingly, animal data 112 in planning data 102 may include a plurality of collections of animal data 206.

In some embodiments, animal data 206 may include information relevant to estimating a veterinary service appointment duration, generating a schedule of veterinary service appointments, and/or generating a route for a provider of veterinary services. For example, temperament may include that an animal is nervous and/or uncooperative with service providers. In another example, habits may include that an animal client typically requires additional, unscheduled, services once an appointment has begun. Additionally, environment data 226 may be include information relevant to estimating a service time (i.e., service duration) for a visit and/or determining service routes. In some embodiments, environment data 226 may include location data, such as location type or geographical information. Environment data 226 may include one or more environmental factors corresponding to an animal, such as their enclosure. For example, environment data 226 may indicate that the animal resides in a large pasture or a mountainous enclosure. In such examples, animals in a large pasture or mountainous enclosure may be more difficult to access, leading to additional appointment time. In another example, environment data 226 may indicate other animals that reside in an enclosure with the animal.

Referring to FIG. 2D, provider data 208 is shown. In the illustrated embodiment, provider data 208 includes provider information 228 and equipment associations 230. Provider information 228 may include data relevant to the provider that is utilized or collected for various aspects of providing veterinary services. For example, provider information 228 may include personal information, skills, abilities, certifications, legal zones, service zones (primary, secondary, etcetera), location data, preferences, habits, tendencies, and the like. Provider information 228 may be generated for each client in the system. Accordingly, provider data 114 in planning data 102 may include a plurality of collections of provider data 208.

In many embodiments, provider data 208 may include information relevant to estimating a veterinary service appointment duration, generating a schedule of veterinary service appointments, and/or generating a route for a provider of veterinary services. For example, tendencies may include that a provider typically has long conversations with clients or drives a certain speed (e.g., slow, or fast). In another example, habits may include that a provider typically adds, or recommends, additional services once an appointment has begun. Equipment associations 230 may include information regarding the tools and equipment that a provider has access to, which can play a role in determining the types of services a provider can perform. Additionally, the tools and equipment can play a role in the amount of time a provider takes to perform a service. For example, specialized tools for specific services, although not required, may reduce the time to perform the service. In a further example, a specialized tool may include a service van with a surgical table in the back. Accordingly, many services may be performed quicker on the surgical table versus in a field.

Referring to FIG. 2E, zone data 210 is shown. In the illustrated embodiment, zone data 210 includes service zones 232 and legal zones 234. The service zones 232 may relate to different service areas covered by a provider. In some embodiments, service zones 232 may overlap. The legal zones 234 may refer to different legal regulations associated with different areas. For example, legal zones 234 may be different for different states, counties, and/or cities based on the local regulations. Legal zones 234 may be associated with providers based on their certifications, training, licenses, and the like. Accordingly, appointments may be assigned to different providers based, at least in part, on zone data 210. In some embodiments, providers may be able to operate in multiple legal zones 234. Accordingly, service zones 232 may span over multiple legal zones 234. Likewise, legal zones 234 may span over multiple service zones 232. In some embodiments, a provider may operate in multiple service zones 232. For example, due to demand (e.g., emergency appointments or excessive demand for services), a service provider route may cross multiple service zones 232. In various embodiments, priority levels associated with appointments may be factored into determining whether a provider is routed into additional service zones.

Referring to FIG. 2F, historical data 246 is shown. In the illustrated embodiment, historical data 246 includes appointment 236a and appointment 236b. However, it will be appreciated that historical data 246 may include any number of historical appointments. Appointment 236a includes appointment data 238a, duration 240a, location data 242a, client data 244a, animal data 248a, and provider data 250a and appointment 236b includes appointment data 238b, duration 240b, location data 242b, client data 244b, animal data 248b, and provider data 250a. In various embodiments, the historical data 246 may be utilized to train an artificial intelligence algorithm to estimate a veterinary service appointment duration, generate a schedule of veterinary service appointments, and/or generate a route for a provider of veterinary services. For example, the artificial intelligence algorithm may include a machine learning algorithm such as a neural network or deep learning algorithm.

In some embodiments, different historical appointments may include different amounts or types of information. For example, some historical appointments may only include a duration and an animal type. In another example, some historical appointments may include manually entered appointment durations while other historical appointments include appointment durations that are automatically determined, such as based on GPS data from a vet service provider computing device. More generally, each appointment in historical data 246 may include one or more aspects, or portions, of data described hereby. In some embodiments, the schedule administrator may collect, automatically or by request, various data items. In various embodiments, the schedule administrator may extract historical appointment data, such as from client reviews or provider feedback.

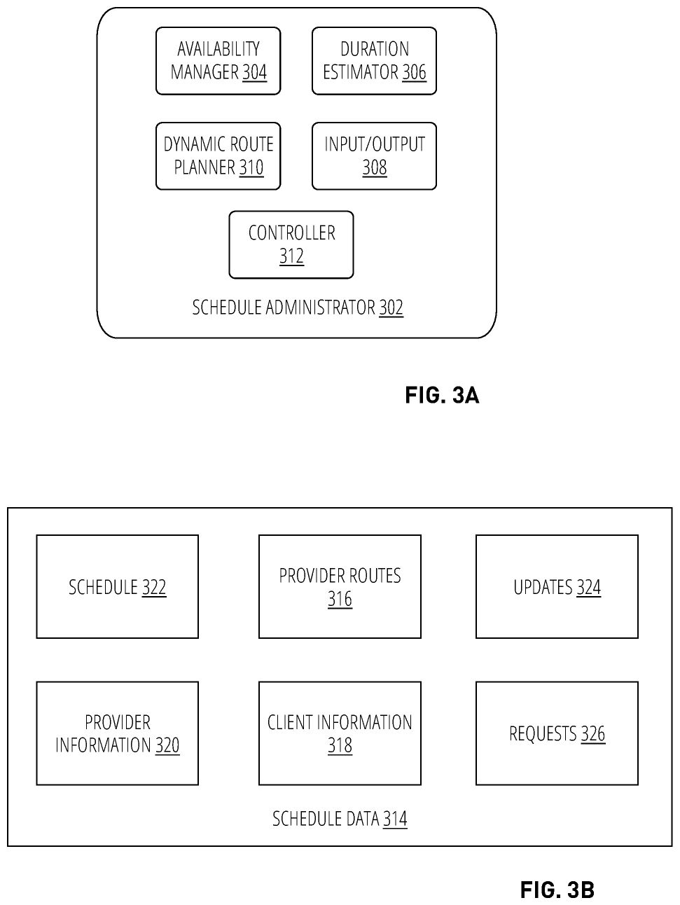

FIG. 3A illustrates various aspects of a schedule administrator 302 according to one or more embodiments described hereby. In the illustrated embodiment, schedule administrator 302 includes availability manager 304, duration estimator 306, dynamic route planner 310, input/output (I/O) 308, and controller 312. Generally, availability manager 304 may determine available service providers for various requested appointments and output it as availability data. Duration estimator 306 may estimate appointment durations, such as using an artificial intelligence algorithm. Dynamic route planner 310 may generate service provider routes, such as using an artificial intelligence algorithm. I/O 308 may facilitate communication for the schedule administrator 302, such as by collecting data (e.g., client data, provider data, zone data, animal data, appointment data, historical data, etcetera), receiving of appointment requests, client communication (e.g., appointment updates, lab results, data collection, etcetera), generating user interfaces, and provider communication (e.g., route updates, lab results, data collection, etcetera). Controller 312 may generally direct control of the different components of the schedule administrator 302 to produce schedule data (e.g., schedule data 314). For example, controller 312 may identify information needed as input and utilize I/O 308 to collect the information. In another example, controller 312 may provide availability data generated by availability manager 304 to duration estimator 306 for estimating appointment duration. In a further example, controller 312 may provide estimated service times (i.e., estimated appointment durations) to the dynamic route planner 310 for generation of provider routes. In many embodiments, schedule administrator 302 may determine whether a requested service is to be performed as a remote, virtual, in-home, or hybrid virtual-in-home veterinary service (different classifications of service). In various embodiments, schedule administrator 302 may request to change appointments between different classifications, such as to improve efficiency or enable additional appointments to be scheduled. In some embodiments, FIG. 3A may include one or more components that are the same or similar to one or more other components of the present disclosure. Further, one or more components of the present disclosure, or aspects thereof, may be incorporated into, or excluded from, the embodiment of FIG. 3A without departing from the scope of this disclosure.

FIG. 3B illustrates various aspects of schedule data 314 according to one or more embodiments described hereby. In the illustrated embodiment, schedule data 314 includes schedule 322, provider routes 316, provider information 320, client information 318, requests 326. In many embodiments, schedule data 314 may be generated and dynamically updated by schedule administrator 302. The schedule 322 may include a schedule of appointments for a period of time, such as a day or a week. The provider routes 316 may include routes for different providers for a period of time, such as a day or a week. In some embodiments, the period of time covered by the schedule 322 may be commensurate with the period of time covered by the provider routes 316. Provider information 320 may include various information regarding various providers that is relevant to the schedule data 314. For example, provider information 320 may include contact information and current (or last known) locations for providers. Similarly, client information 318 may include information regarding various clients and/or animals that is relevant to the schedule data 314. For example, client information 318 may include contact information, addresses for clients, and/or animal medical data. The updates 324 may include various changes that have been made to the schedule 322 and/or provider routes 316, such as delays and/or additional appointments. For example, updates 324 may include a log of changes that have occurred. In some embodiments, updates 324 may be based on GPS data. For example, GPS data may be utilized to determine a veterinary service provider is behind schedule. The requests 326 may include received and/or sent requests. For example, requests 326 may include original appointment requests corresponding to appointments on schedule 322. In another example, requests 326 may include sent and/or outstanding requests for data, such as data from providers and/or clients. In a further example, requests 326 may include a pending request sent to a client to reschedule an appointment or convert an appointment from a first class to a second claims (e.g., from in-home to virtual) hybrid virtual-in-home veterinary service. In yet another example, requests 326 may include a pending request sent to a provider to accept an updated route. In some embodiments, FIG. 3B may include one or more components that are the same or similar to one or more other components of the present disclosure. Further, one or more components of the present disclosure, or aspects thereof, may be incorporated into, or excluded from, the embodiment of FIG. 3B without departing from the scope of this disclosure.

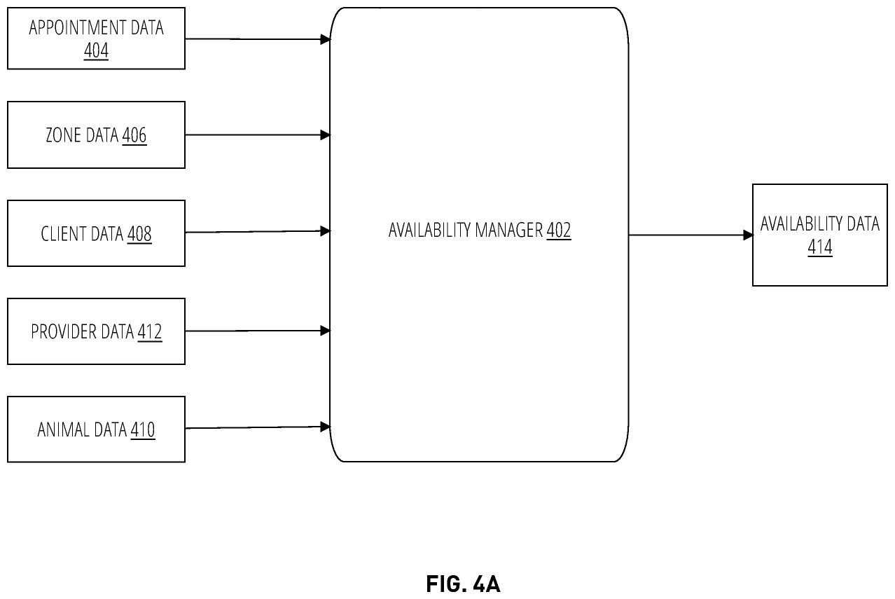

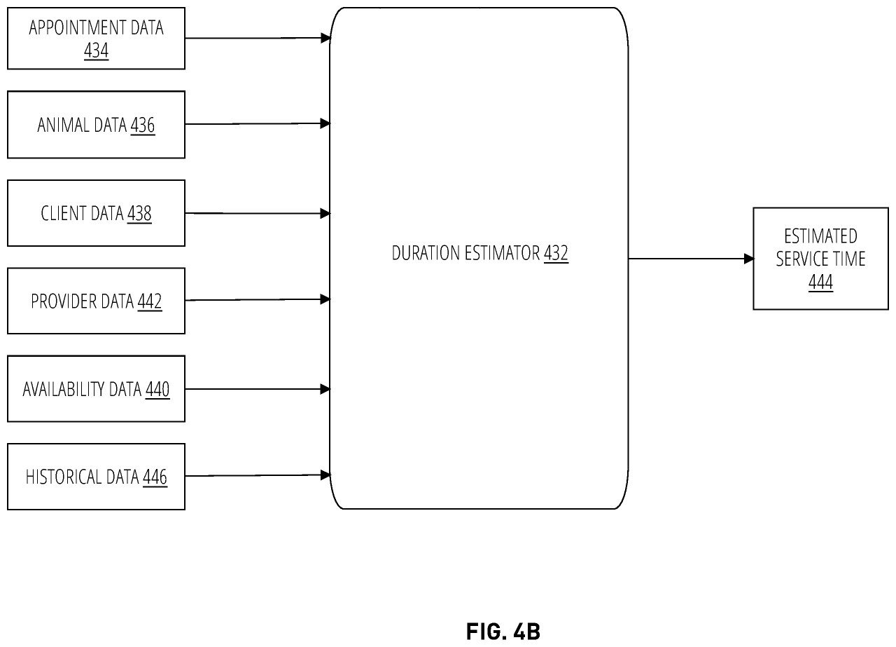

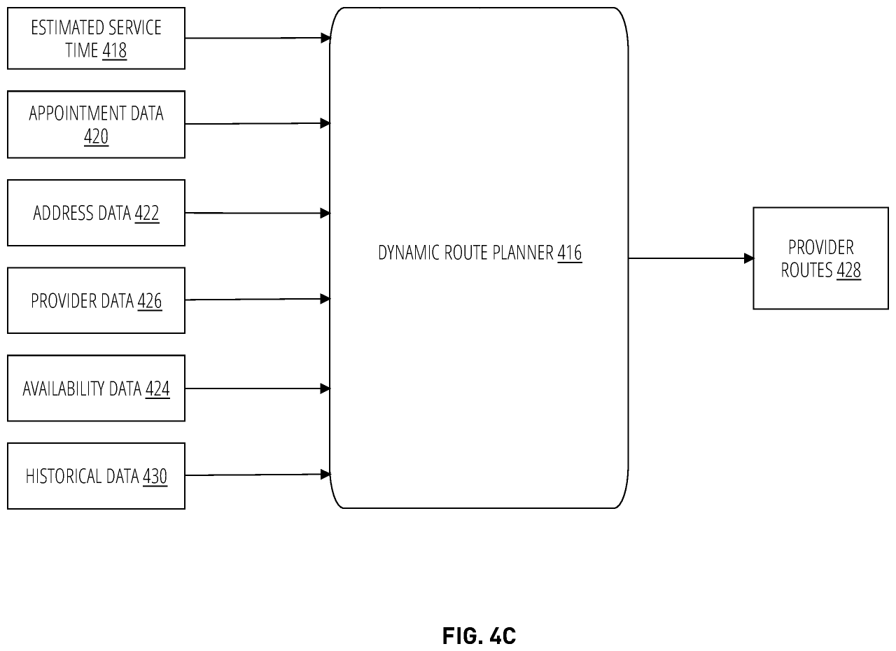

FIG. 4A illustrates an exemplary process flow for an availability manager 402 according to one or more embodiments described hereby. In the illustrated embodiment, availability manager 402 takes appointment data 404, zone data 406, client data 408, provider data 412, and animal data 410 as input and generates availability data 414 as output. In other embodiments, additional, alternative, or less information may be provided to availability manager 402 as input. Zone data 406 may be the same as, or a portion of, zone data 210. Similarly, other inputs may be the same as, or a portion of, other data items of the same name. In various embodiments, availability data 414 may include available providers for each appointment. In many embodiments, availability manager 402 may utilize an artificial intelligence algorithm to generate availability data 414. In many such embodiments, the artificial intelligence algorithm may be trained on historical data (e.g., historical data 246). In several embodiments, the data provided as input to availability manager 402 (and/or utilized for training the artificial intelligence algorithm) may be in the form of a multi-dimensional vector. In some embodiments, FIG. 4A may include one or more components that are the same or similar to one or more other components of the present disclosure. For example, appointment data 404 may include a subset of appointment data 108. Further, one or more components of the present disclosure, or aspects thereof, may be incorporated into, or excluded from, the embodiment of FIG. 4A without departing from the scope of this disclosure. For example, historical appointment data 236b may be incorporated without departing from the scope of this disclosure.

FIG. 4B illustrates an exemplary process flow for a duration estimator 432 according to one or more embodiments described hereby. In the illustrated embodiment, duration estimator 432 takes appointment data 434, animal data 436, client data 438, provider client data 438, availability data 440, and historical data 446 as input and generates estimated service time 444 (i.e., an estimated appointment duration). In other embodiments, additional, alternative, or less information may be provided to duration estimator 432 as input. Availability data 440 may be the same as, or a portion of, availability data 414. Similarly, other inputs may be the same as, or a portion of, other data items of the same name. In various embodiments, estimated service time 444 may include an estimated duration of an appointment identified in appointment data 434. In some embodiments, estimated service time 444 may include transit times. In some embodiments, FIG. 4B may include one or more components that are the same or similar to one or more other components of the present disclosure. For example, historical data 446 may include a subset of historical data 118. Further, one or more components of the present disclosure, or aspects thereof, may be incorporated into, or excluded from, the embodiment of FIG. 4A without departing from the scope of this disclosure. For example, client data 438 may be excluded without departing from the scope of this disclosure.

In many embodiments, duration estimator 432 may utilize an artificial intelligence algorithm, such as a machine learning algorithm, to generate estimated service time 444. In many such embodiments, the artificial intelligence algorithm may be trained on historical data 446. In one embodiment, historical data 446 may be the same or similar to appointment 236a of historical data 246. In some embodiments, the data provided as input to duration estimator 432 (and/or utilized for training the artificial intelligence algorithm) may be in the form of a multi-dimensional vector. For example, the multi-dimensional vector may include a first value generated based on a veterinary service type included in appointment data 434, a second value generated based on the animal type included in animal data 436, and a third value generated based on the location type included in one or more of appointment data 434, client data 438, and animal data 436. Accordingly, in the previous example, the multi-dimensional vector may include a veterinary service type dimension, an animal type dimension, and a location type dimension. It will be appreciated that any combination of dimensions based on available data may be used for a multi-dimensional vector utilized in conjunction with duration estimator 432 without departing from the scope of this disclosure. In many embodiments, analogous data included in historical data 446 (e.g., location type in prior appointments included in historical data 446, etcetera) may be utilized for training the artificial intelligence algorithm. In some embodiments, FIG. 4B may include one or more components that are the same or similar to one or more other components of the present disclosure. For example, historical data 446 may include a subset of historical data 118. Further, one or more components of the present disclosure, or aspects thereof, may be incorporated into, or excluded from, the embodiment of FIG. 4B without departing from the scope of this disclosure. For example, client data 438 may be excluded without departing from the scope of this disclosure.

FIG. 4C illustrates an exemplary process flow for a dynamic route planner 416 according to one or more embodiments described hereby. In the illustrated embodiment, dynamic route planner 416 takes estimated service time 418, appointment data 420, address data 422, provider data 426, availability data 424, and historical data 430 as input and generates provider routes 428. In other embodiments, additional, alternative, or less information may be provided to dynamic route planner 416 as input. Estimated service time 418 may be the same as, or a portion of, estimated service time 444. Similarly, other inputs may be the same as, or a portion of, other data items of the same name. Further, address data 422 may be a portion of client data 408 or client data 438. In various embodiments, provider routes 428 may include instructions for each scheduled appointment. In some embodiments, provider routes 428 may include transit times. For example, transit times may be determined based on address data 422. In some embodiments, FIG. 4C may include one or more components that are the same or similar to one or more other components of the present disclosure. For example, historical data 430 may include a subset of historical data 118. Further, one or more components of the present disclosure, or aspects thereof, may be incorporated into, or excluded from, the embodiment of FIG. 4C without departing from the scope of this disclosure.

In many embodiments, dynamic route planner 416 may utilize an artificial intelligence algorithm, such as a machine learning algorithm, to generate provider routes 428. In many such embodiments, the artificial intelligence algorithm may be trained on historical data 430. In one embodiment, historical data 430 may be the same or similar to appointment 236a of historical data 246. In some embodiments, the data provided as input to dynamic route planner 416 (and/or utilized for training the artificial intelligence algorithm) may be in the form of a multi-dimensional vector. It will be appreciated that any combination of dimensions based on available data may be used for a multi-dimensional vector utilized in conjunction with dynamic route planner 416 without departing from the scope of this disclosure. In many embodiments, analogous data included in historical data 430 (e.g., transit times between different locations in prior appointments included in historical data 446, etcetera) may be utilized for training the artificial intelligence algorithm.

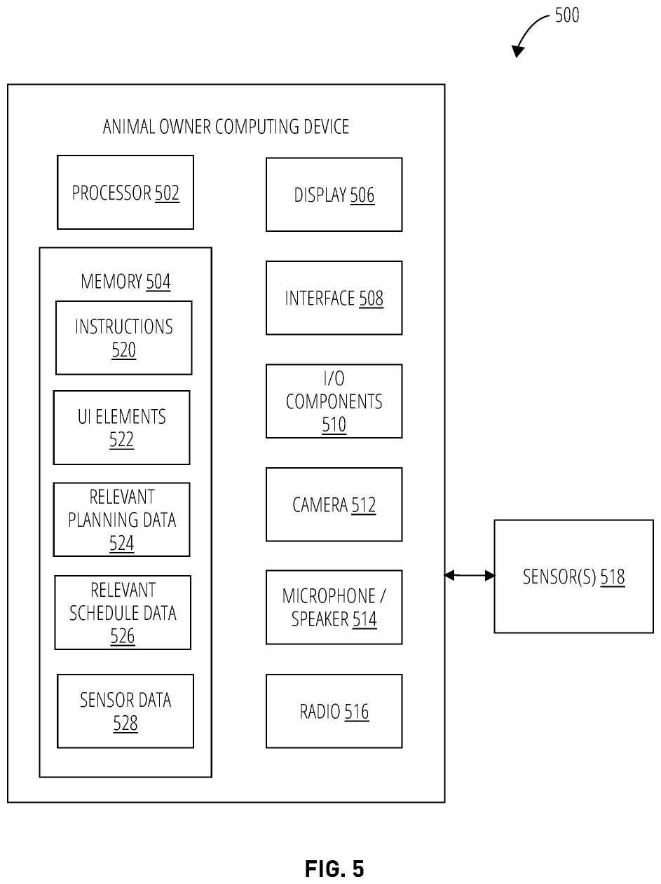

FIGS. 5-7B illustrate devices and systems arranged for facilitating and/or providing veterinary services. Turning more particularly to FIG. 5, which illustrates an example client computing device 500 (often simply device 500), which can be a computing device owned and/or operated by an owner of an animal to request or that is to receive veterinary medicine as detailed herein. In general, device 500 can be any of a variety of computing devices, such as, a smart phone, a tablet computer, a laptop computer, a desktop computer, a smart watch, a smart pet collar, or the like. Device 500 includes a processor 502, a memory 504, a display 506, an interface 508, input and output (I/O) components 510, camera 512, microphone and speaker 514, and radio 516. Device 500 may optionally also include, or be operably coupled to, one or more sensors 518.

The memory 504 may store instructions 520 executable by processor 502, which when executed cause processor 502 and device 500 to implement or carry out features and functions detailed herein. Memory 504 may further store UI elements 522, relevant planning data 524, relevant schedule data 526, and sensor data 528. With some examples, processor 502 can execute instructions 520 to generate user interface (UI) elements 522 and relevant planning data 524. Additionally, processor 502 can execute instructions 520 to retrieve relevant schedule data 526. Furthermore, processor 502 can execute instructions 520 to receive sensor data 528 from sensor 518.

Device 500 can be used to facilitate other embodiments of the present disclosure, such as, for example, a remote, virtual, in-home, or hybrid virtual-in-home veterinary appointment, including selecting the appointment, scheduling the appointment, conducting the appointment, following up on the appointment (e.g., ordering prescriptions, or the like).

With some examples, the processor 502 may include circuitry or processor logic, such as, for example, any of a variety of commercial processors. In some examples, the processor 502 may include multiple processors, a multi-threaded processor, a multi-core processor (whether the multiple cores coexist on the same or separate dies), and/or a multi-processor architecture of some other variety by which multiple physically separate processors are in some way linked. Additionally, in some examples, the processor 502 may include graphics processing portions and may include dedicated memory, multiple-threaded processing and/or some other parallel processing capability.

The memory 504 may include logic, a portion of which includes arrays of integrated circuits, forming non-volatile memory to persistently store data or a combination of non-volatile memory and volatile memory. It is to be appreciated, that the memory 504 may be based on any of a variety of technologies. In particular, the arrays of integrated circuits included in memory 504 may be arranged to form one or more types of memory, such as, for example, dynamic random access memory (DRAM), NAND memory, NOR memory, or the like.

Display 506 can be based on any of a variety of display technologies, such as, for example, a cathode ray tube (CRT), a liquid crystal display (LCD), plasma display, light emitting diode (LED) display, or an organic light emitting diode (OLED) display. With some examples, display 506 can be a touch sensitive display. It is noted, display 506 may be external to device 500, such as, for example, embodied as a computer monitor or television and coupled to device 500 via any of a variety of display data interfaces.

Interface 508 may include logic and/or features to support a communication interface. For example, the interface 508 may include one or more interfaces that operate according to various communication protocols or standards to communicate over direct or network communication links. Direct communications may occur via use of communication protocols or standards described in one or more industry standards (including progenies and variants). For example, the interface 508 may facilitate communication over a bus, such as, for example, peripheral component interconnect express (PCIe), non-volatile memory express (NVMe), universal serial bus (USB), system management bus (SMBus), SAS (e.g., serial attached small computer system interface (SCSI)) interfaces, serial AT attachment (SATA) interfaces, or the like.

The I/O components 510 may include one or more components to provide input to or to provide output from the device 500. For example, the I/O components 510 may be a keyboard (hardware, virtual, etc.), mouse, joystick, track pad, button, touch layers of a display, haptic feedback device, or the like.

Camera 512 as well as microphone and speaker 514 may be any of a variety of cameras, microphone, and speakers used for teleconference and video conference applications. Camera 512 and microphone and speaker 514 may be integrated into device 500 or may be external to device 500.

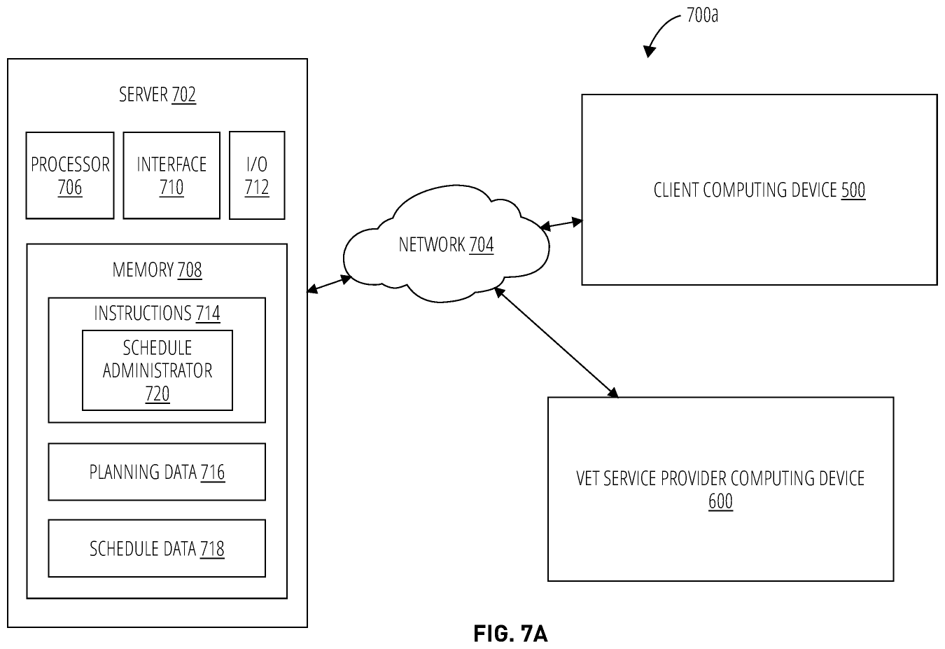

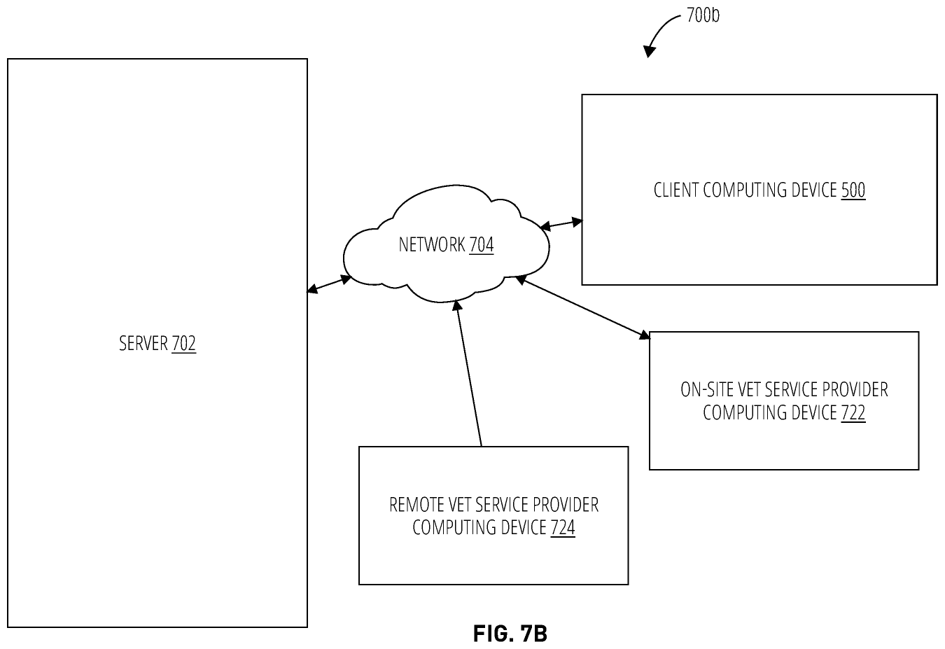

The radio 516 may include circuitry arranged to communicate data with one or more other devices (see FIG. 7A) via any of a variety of communication protocols. Such communication may involve communication across one or more networks, such a wireless local area networks (WLAN) and/or cellular network. In some examples, radio 516 can be arranged to communicate via Wi-Fi, Bluetooth, Zigbee, LTE, 5G, RFID, or the like.

The sensor 518 (or sensors 518) may include a number of any of a variety of sensors arranged to detect information, such, as, physical surrounding information, geo-information, biometric information, or the like. For example, sensor 518 can include a thermometer, a heart rate sensor, a digital stethoscope, a digital otoscope, a digital ophthalmoscope, a blood pressure monitor, a tonopen, an ultrasound, a centrifuge, a cold laser, a multiscope (e.g., touch screen multiscope with telemedicine capabilities, or the like), an x-ray, a portable lab device, a refractometer, or the like. Signals from sensor 518 can be used to capture indications of lab results, biometric indications, or the like of a pet. It is noted, that sensor 518 could be located externally to device 500. For example, sensor 518 could be embodied in a smart pet, a microchip implanted in a pet, or the like. Such a sensor 518 could be wirelessly coupled to device 500.

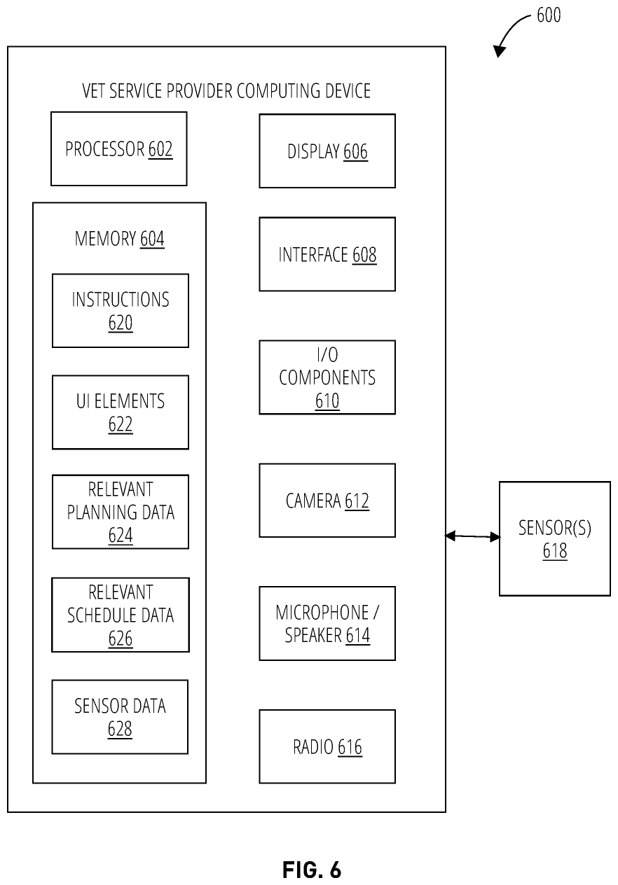

FIG. 6 illustrates vet service provider computing device 600 (often simply device 600), which can be a computing device owned and/or operated by a veterinary medicine service provider. During use, device 600 may be operated by an administrator of a veterinary service provider, a veterinary technician, a nurse, or a veterinarian. In some embodiments, vet service provider computing device 600 may implement one or more aspects of schedule administrator 104. In general, device 600 can be any of a variety of computing devices, such as, a smart phone, a tablet computer, a laptop computer, a desktop computer, or the like. Device 600 includes, at least, a processor 602, a memory 604, a display 606, an interface 608, I/O components 610, camera 612, microphone and speaker 614, and radio 516. Device 600 may optionally also include, or be operably coupled to, one or more sensors 618.

The memory 604 may store instructions 620 executable by processor 602, which when executed cause processor 602 and device 600 to implement or carry out features and functions detailed herein. Memory 604 may further store UI elements 622, relevant planning data 624, relevant schedule data 626, and sensor data 628. With some examples, processor 602 can execute instructions 620 to generate user interface (UI) elements 622 and relevant schedule data 626. Additionally, processor 602 can execute instructions 620 to retrieve relevant planning data 624 and relevant schedule data 526. Furthermore, processor 602 can execute instructions 620 to receive sensor data 628 from sensor 618. In some examples, sensor data 628 can be received from device 500 (e.g., via server 702, or the like).

Device 600 can be used to facilitate other embodiments of the present disclosure, such as, for example, a remote, virtual, in-home, and/or hybrid virtual-in-home veterinary appointment, including identifying the appointment scheduled via device 500, conducting the appointment, documenting the appointment, etc.

With some examples, the processor 602 may include circuitry or processor logic, such as, for example, any of a variety of commercial processors. In some examples, the processor 602 may include multiple processors, a multi-threaded processor, a multi-core processor (whether the multiple cores coexist on the same or separate dies), and/or a multi-processor architecture of some other variety by which multiple physically separate processors are in some way linked. Additionally, in some examples, the processor 602 may include graphics processing portions and may include dedicated memory, multiple-threaded processing and/or some other parallel processing capability.

The memory 604 may include logic, a portion of which includes arrays of integrated circuits, forming non-volatile memory to persistently store data or a combination of non-volatile memory and volatile memory. It is to be appreciated, that the memory 604 may be based on any of a variety of technologies. In particular, the arrays of integrated circuits included in memory 604 may be arranged to form one or more types of memory, such as, for example, dynamic random access memory (DRAM), NAND memory, NOR memory, or the like.

Display 606 can be based on any of a variety of display technologies, such as, for example, a cathode ray tube (CRT), a liquid crystal display (LCD), plasma display, light emitting diode (LED) display, or an organic light emitting diode (OLED) display. With some examples, display 606 can be a touch sensitive display. It is noted, display 606 may be external to device 600, such as, for example, embodied as a computer monitor or television and coupled to device 600 via any of a variety of display data interfaces.

Interface 608 may include logic and/or features to support a communication interface. For example, the interface 608 may include one or more interfaces that operate according to various communication protocols or standards to communicate over direct or network communication links. Direct communications may occur via use of communication protocols or standards described in one or more industry standards (including progenies and variants). For example, the interface 608 may facilitate communication over a bus, such as, for example, peripheral component interconnect express (PCIe), non-volatile memory express (NVMe), universal serial bus (USB), system management bus (SMBus), SAS (e.g., serial attached small computer system interface (SCSI)) interfaces, serial AT attachment (SATA) interfaces, or the like.

The I/O components 610 may include one or more components to provide input to or to provide output from the device 500. For example, the I/O components 610 may be a keyboard (hardware, virtual, etc.), mouse, joystick, track pad, button, touch layers of a display, haptic feedback device, or the like.

Camera 612 as well as microphone and speaker 614 may be any of a variety of cameras, microphone, and speakers used for teleconference and video conference applications. Camera 612 and microphone and speaker 614 may be integrated into device 600 or may be external to device 600.

The radio 616 may include circuitry arranged to communicate data with one or more other devices (see FIG. 7A) via any of a variety of communication protocols. Such communication may involve communication across one or more networks, such a wireless local area networks (WLAN) and/or cellular network. In some examples, radio 616 can be arranged to communicate via Wi-Fi, Bluetooth, Zigbee, LTE, 5G, or the like.

The sensor 618 (or sensors 618) may include a number of any of a variety of sensors arranged to detect information, such, as, physical surrounding information, geo-information, biometric information, or the like. For example, sensor 618 can include a radar sensor, infrared sensors, light sensors, RFID sensors, gyroscopes, a global positioning sensors (GPS), a heart rate sensor, a temperature sensor, or the like. Signals from sensor 618 can be used to collect animal data or other data relevant to facilitate the provision of veterinary services. It is noted, that sensor 618 could be located externally to device 600.

The sensor 618 (or sensors 618) may include a number of any of a variety of sensors arranged to detect information, such, as, physical surrounding information, geo-information, biometric information, or the like. For example, sensor 618 can include a thermometer, a heart rate sensor, a digital stethoscope, a digital otoscope, a digital ophthalmoscope, a blood pressure monitor, a tonopen, an ultrasound, a centrifuge, a cold laser, a multiscope (e.g., touch screen multiscope with telemedicine capabilities, or the like), an x-ray, a portable lab device, a refractometer, or the like. Signals from sensor 618 can be used to capture indications of lab results, biometric indications, or the like of a pet. It is noted, that sensor 618 could be located externally to device 600. In other examples, device 600 could be communicatively connected to sensor 518 of device 500 (e.g., via server 702 and network 704, or the like).

FIG. 7A illustrates an example system 700a including device 500, device 600, and server 702. In system 700a, device 500, device 600, and server 702 are communicatively coupled via network 704. Network 704 could be, for example, a local area network (LAN), a wide area network (WAN), or a cellular network (e.g., LTE, 3GPP, or the like). In some embodiments, network 704 could include the Internet.

The server 702 can include a processor 706, memory 708, interface 710, and I/O components 712. The memory 708 may store instructions 714 with schedule administrator 720, planning data 716, and schedule data 718. With some examples, processor 706 can execute instructions 714 to generate, receive, and/or provide planning data 716 and schedule data 718. Additionally, processor 706 can execute instructions 714 to implement schedule administrator 720 (which may be the same as schedule administrator 104 or schedule administrator 302). For example, processor 706 can execute instructions 714 to coordinate scheduling of a remote, virtual, in-home, and/or hybrid virtual-in-home veterinary service via interaction with device 500 and/or device 600. Likewise, processor 706 can execute instructions 714 to provide details (e.g., via schedule data 718) of a scheduled remote, virtual, in-home, and/or hybrid virtual-in-home veterinary service to device 600. Server 702 within system 700a can be used to facilitate other embodiments of the present disclosure, such as, for example, schedule administrator 302, availability manager 402, duration estimator 432, and/or dynamic route planner 416. These, and other embodiments, are described in greater detail below.

With some examples, the processor 706 may include circuitry or processor logic, such as, for example, any of a variety of commercial processors. In some examples, the processor 706 may include multiple processors, a multi-threaded processor, a multi-core processor (whether the multiple cores coexist on the same or separate dies), and/or a multi-processor architecture of some other variety by which multiple physically separate processors are in some way linked. Additionally, in some examples, the processor 706 may include graphic s processing portions and may include dedicated memory, multiple-threaded processing and/or some other parallel processing capability.

Memory 708 may include logic, a portion of which includes arrays of integrated circuits, forming non-volatile memory to persistently store data or a combination of non-volatile memory and volatile memory. It is to be appreciated, that the memory 708 may be based on any of a variety of technologies. In particular, the arrays of integrated circuits included in memory 708 may be arranged to form one or more types of memory, such as, for example, dynamic random access memory (DRAM), NAND memory, NOR memory, or the like.

Interface 710 may include logic and/or features to support a communication interface. For example, the interface 710 may include one or more interfaces that operate according to various communication protocols or standards to communicate over direct or network communication links. Direct communications may occur via use of communication protocols or standards described in one or more industry standards (including progenies and variants). For example, the interface 710 may facilitate communication over a bus, such as, for example, peripheral component interconnect express (PCIe), non-volatile memory express (NVMe), universal serial bus (USB), system management bus (SMBus), SAS (e.g., serial attached small computer system interface (SCSI)) interfaces, serial AT attachment (SATA) interfaces, or the like. Additionally, interface 710 can be a network interface arranged to couple server 702 to a network (e.g., network 704).

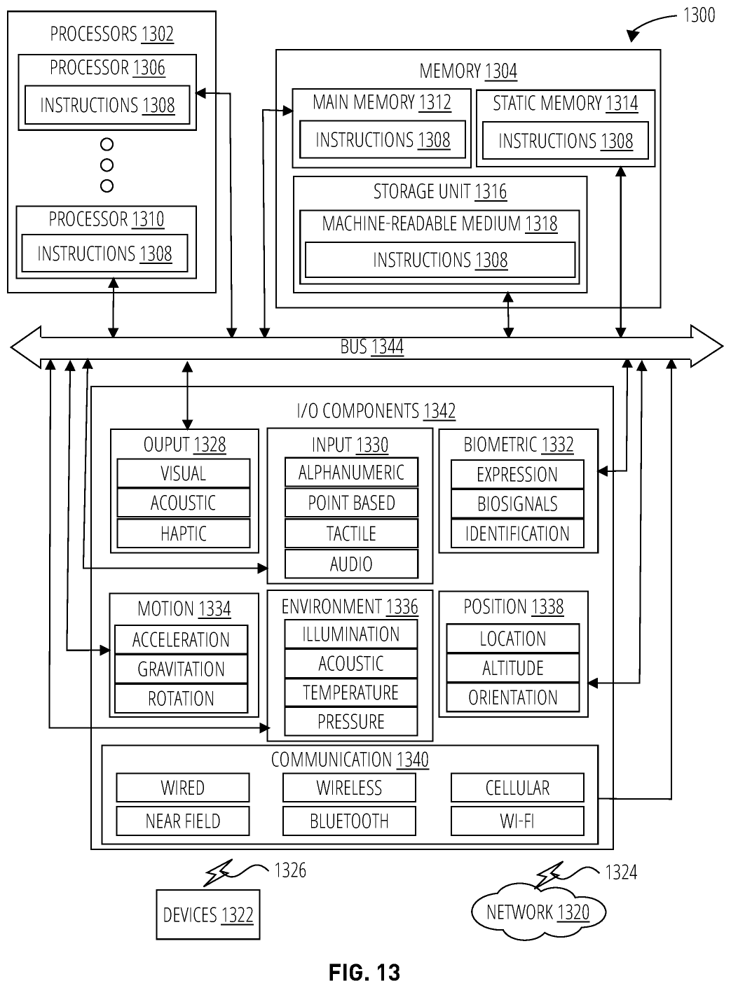

As noted, system 700a can facilitate numerous embodiments to facilitate veterinary services. Accordingly, operation and features of system 700a are described with reference other figures depicted and described herein, such as, for example, FIG. 7B or FIG. 13.

FIG. 7B illustrates an example system 700a including device 500 and server 702. Additionally, system 700a includes multiple vet service provider computing devices. For example, system 700a includes on-site vet service provider computing device 722 and remote vet service provider computing device 724. In general, device 722 and device 724 can be like device 600 of FIG. 6. In system 700a, device 500, device 722, device 724, and server 702 are communicatively coupled via network 704. Network 704 could be, for example, a local area network (LAN), a wide area network (WAN), or a cellular network (e.g., LTE, 3GPP, or the like). In some embodiments, network 704 could include the Internet.

System 700b may facilitate a hybrid virtual-in-home veterinarian visit. For example, system 700b may facilitate video conferencing or remote attendance by a veterinarian via device 724 to an in-home veterinarian visit conducted by a nurse via device 722. For example, during operation, a nurse can use on-site vet service provider computing device 722 to receive relevant planning data 624, update relevant planning data 624, receive relevant schedule data 626, and/or update relevant schedule data 626 as part of conducting a hybrid virtual-in-home veterinary visit. In particular processor 602 can execute instructions 620 to receive relevant planning data 624, update relevant planning data 624, receive relevant schedule data 626, and/or update relevant schedule data 626 as part of conducting a hybrid virtual-in-home veterinary visit as described herein. Likewise, processor 602 can execute instructions 620 to conduct a virtual meeting with a veterinarian (e.g., via remote vet service provider computing device 724) during a hybrid virtual-in-home visit.

Additionally, during operation, a veterinarian can use remote vet service provider computing device 724 to receive relevant planning data 624, update relevant planning data 624, receive relevant schedule data 626, and/or update relevant schedule data 626 as part of conducting a hybrid virtual-in-home veterinary visit. In particular processor 602 can execute instructions 620 to receive relevant planning data 624, update relevant planning data 624, receive relevant schedule data 626, and/or update relevant schedule data 626 as part of conducting a hybrid virtual-in-home veterinary visit as described herein. Likewise, processor 602 can execute instructions 620 to attend a virtual meeting with a nurse (e.g., via on-site vet service provider computing device 722) during a hybrid virtual-in-home visit.

In some examples, components in the system 700b (or 700a) may record a remote, virtual, or hybrid virtual-in-home veterinary exam, as well as other relevant data such as transit times, and save the as historical data. In a further example, indications of sensor data 528 and/or sensor data 628 may be saved to historical data. In one embodiment, such recordings or sensor data may be added to animal medical records. In some embodiments, system 700b (or 700a) may identify audio and/or speech from recording or live veterinary exams (e.g., remote, virtual, hybrid virtual-in-home, etc.), transcribe the speed, and add save the transcript to historical data or medical records.

With some examples, components in the system 700b (or 700a) may include circuitry or processor logic, such as, for example, any of a variety of commercial processors. In some examples, the processor 706 may include multiple processors, a multi-threaded processor, a multi-core processor (whether the multiple cores coexist on the same or separate dies), and/or a multi-processor architecture of some other variety by which multiple physically separate processors are in some way linked. Additionally, in some examples, the processor 706 may include graphics processing portions and may include dedicated memory, multiple-threaded processing and/or some other parallel processing capability.

As noted, system 700b can facilitate numerous embodiments to numerous embodiments to facilitate veterinary services. Operation and features of system 700b are described with reference other figures depicted and described herein, such as, for example, FIG. 13.

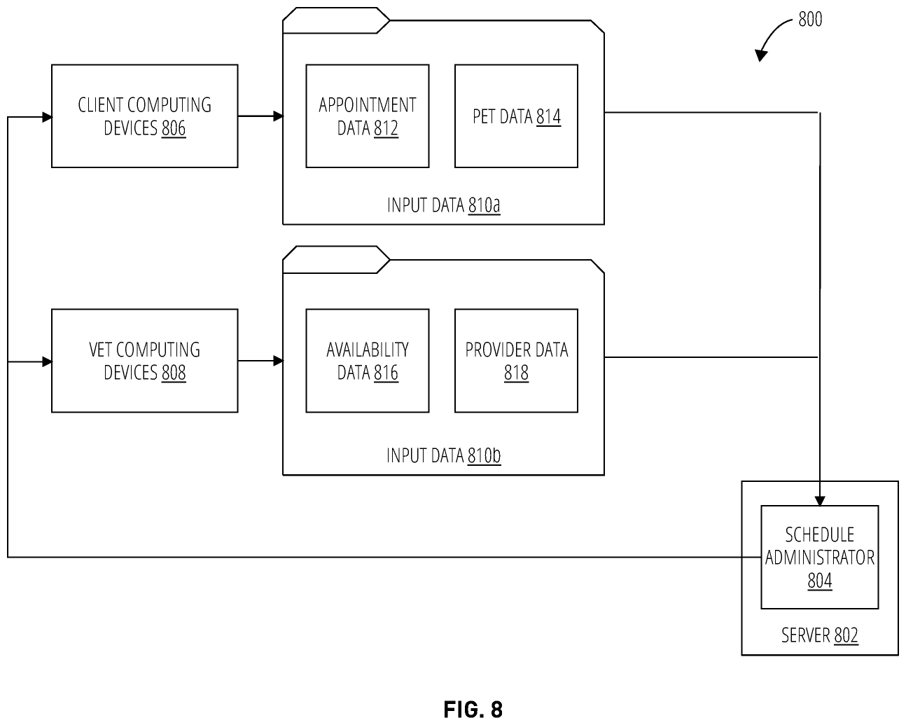

FIG. 8 illustrates an exemplary process flow 800 according to one or more embodiments described hereby. In the illustrated embodiment, process flow 800 includes one or more client computing devices 806 providing input data 810a and one or more vet computing devices 808 providing input data 810b to schedule administrator 804 within server 802. The input data 810a provided by the one or more client computing devices 806 may include appointment data 812 and pet data 814. The input data 810b provided by the one or more vet computing devices 808 may include availability data 816 and provider data 818. In some embodiments, FIG. 8 may include one or more components that are the same or similar to one or more other components of the present disclosure. For example, schedule administrator 804 maybe the same or similar to schedule administrator 302. Further, one or more components of the present disclosure, or aspects thereof, may be incorporated into, or excluded from, the embodiment of FIG. 8 without departing from the scope of this disclosure.

In various embodiments, schedule administrator 804 may generate one or more of a schedule, a route, and one or more time estimates based on one or more portions of the input data 810a provided via client computing devices 806 and the input data 810a provided via vet computing devices 808. For example, schedule administrator 804 may generate one or more routes and send them to one or more vet computing devices 808. Similarly, schedule administrator 804 may generate one or more schedules and send them to one or more client computing devices 806. Further, the routes and/or schedules may be generated based on duration estimates generated by the schedule administrator 804.

In some embodiments, input data 810a may be provided via a user interface. For example, a client may utilize a client computing device to request an appointment during a specific time block (e.g., 2 hours, 4 hours, etcetera). For example, a client may decide between an Sam to 12 pm block and a 1 μm to 5 pm block. The time block may be determined by schedule administrator 804 based on availability data 816 provided via a vet computing device. For example, availability data 816 may include schedule information regarding one or more veterinary service providers. In some embodiments, the schedule administrator 804 may add a break period between different time blocks.

In many embodiments, traffic data may be collected, including estimated traffic data for future times. For example, traffic data may be collected from a map service. The traffic data may be utilized by the schedule administrator 804 in generating one or more schedules, routes, and/or estimated service times. In many embodiments, notifying clients of a schedule may include identifying a start time, or a narrower time window, within their requested time block. For instance, if a client requested an Sam to 12 pm time block, after the schedules and routes are generated, the client may be notified that their appointment will start at 9 am or that their appointment will begin between 9 am and 10 am.

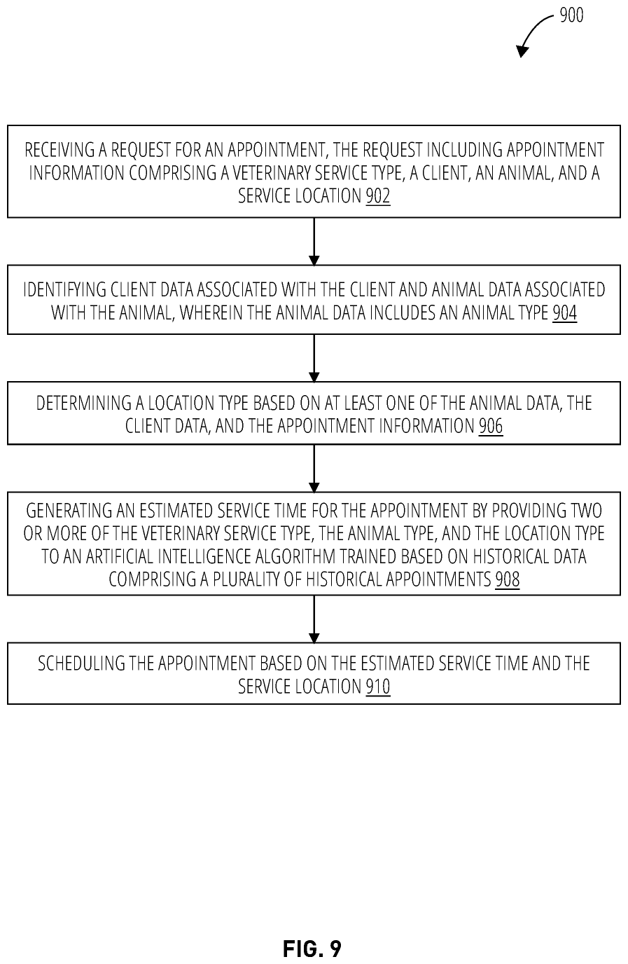

FIG. 9 illustrates one embodiment of a logic flow 900, which may be representative of operations that may be executed in various embodiments in conjunction with techniques disclosed hereby. The logic flow 900 may be representative of some or all of the operations that may be executed by one or more components/devices/environments described hereby, such as schedule administrator 302 and/or duration estimator 432. The embodiments are not limited in this context.

In the illustrated embodiment, logic flow 900 may begin at block 902. At block 902 “receiving a request for an appointment, the request including appointment information comprising a veterinary service type, a client, an animal, and a service location” a request for an appointment may be received. The request may include appointment information comprising a veterinary service type, a client, an animal, and a service location. For example, schedule administrator 302 may receive appointment request 212a via a graphical user interface generated with I/O 308. Further, appointment request 212a may include appointment information 214a identifying a veterinary service type, a client, an animal, and a service location.

Proceeding to block 904 “identifying client data associated with the client and animal data associated with the animal, wherein the animal data includes an animal type” client data associated with the client and animal data including an animal type associated with the animal may be identified. For instance, schedule administrator 104 may identify client data 204 associated with a client identified in appointment request 212b and animal data 206 associated with an animal identified in appointment request 212b. Further, animal information 224 in animal data 206 may include an animal type corresponding to the animal identified in the appointment request 212b. For example, the animal type may include a dog, a cat, a bird, a horse, a snake, a turtle, etcetera. At block 906 “determining a location type based on at least one of the animal data, the client data, and the appointment information” a location type may be determined based on at least one of the animal data, the client data, and the appointment information. For example, schedule administrator 104 may utilize one or more of appointment request 212a, client data 204, and animal data 206 to determine a location type. In some embodiments, the location type may include one or more of a single-family home, a multi-family home, a rural home, a suburban home, and an urban home. In some embodiments, the location type may be determined based on appointment type (e.g., home/same day vs. onsite). For example, a surgical appointment type may be utilized to determine an operating room (e.g., in a van or at an office) as the location type.

Continuing to block 908 “generating an estimated service time for the appointment by providing two or more of the veterinary service type, the animal type, and the location type to an artificial intelligence algorithm trained based on historical data comprising a plurality of historical appointments” an estimated service time for the appointment may be generated by providing two or more of the veterinary service type, the animal type, and the location type to an artificial intelligence algorithm. Further, the artificial intelligence algorithm may be trained based on historical data comprising a plurality of historical appointments. For example, duration estimator 432 may include an artificial intelligence algorithm to generate estimated service time 444 based on two or more portions of appointment data 434, animal data 436, and client data 438. In one embodiment, the estimated service time for the appointment may be generated by providing one or more of the veterinary service type, the animal type, and the location type to an artificial intelligence algorithm.

In many embodiments, the artificial intelligence algorithm may be trained based on a plurality of historical appointments included in historical data 446. In one embodiment, the artificial intelligence algorithm may include a neural network. At block 910 “scheduling the appointment based on the estimated service time and the service location” the appointment may be scheduled based on the estimated service time and the service location. For example, dynamic route planner 310 or controller 312 may schedule the appointment in schedule 322. In some embodiments, controller 312 may generate schedule 322 based on provider routes 428 produced by dynamic route planner 416.

In some embodiments, the dynamic route planner 416 may generate a route for a veterinary service provider based on the estimated service time and the service location. In some such embodiments, the route includes the appointment and at least one additional appointment. In various embodiments, at least one of the plurality of historical appointments utilized to train the artificial intelligence algorithm are associated with, or include, two or more of a corresponding service time, a corresponding veterinary service type, a corresponding animal type, and a corresponding location type. In many embodiments, at least one of the plurality of historical appointments are associated with, or include, a corresponding service provider and two or more of a corresponding service time, a corresponding veterinary service type, a corresponding animal type, and a corresponding location type.

In several embodiments, a service provider may be determined for the appointment by availability manager 402. In several such embodiments, the estimated service time for the appointment may be generated by providing the service provider and the two or more of the veterinary service type, the animal type, and the location type to the artificial intelligence algorithm. In some embodiments, the estimated service time for the appointment may be generated by providing the client data and the two or more of the veterinary service type, the animal type, and the location type to the artificial intelligence algorithm. In various embodiments, the animal type is provided to the artificial intelligence algorithm in animal data including the animal type and data indicative of at least one historical interaction with the animal, such as in animal data 248a.

In some embodiments, a multi-dimensional vector including a first value generated based on the veterinary service type, a second value generated based on the animal type, and a third value generated based on the location type may be generated for the appointment. In some such embodiments, the multi-dimensional vector may be provided to the artificial intelligence algorithm to provide the two or more of the veterinary service type, the animal type, and the location type to the artificial intelligence algorithm for generation of the estimated service time. In many embodiments, zone data comprising a service zone corresponding to the service location and a legal zone corresponding to the service location may be determined based on the service location. In many such embodiments, a service provider may be determined, such as by availability manager 402, for the appointment based on the zone data.

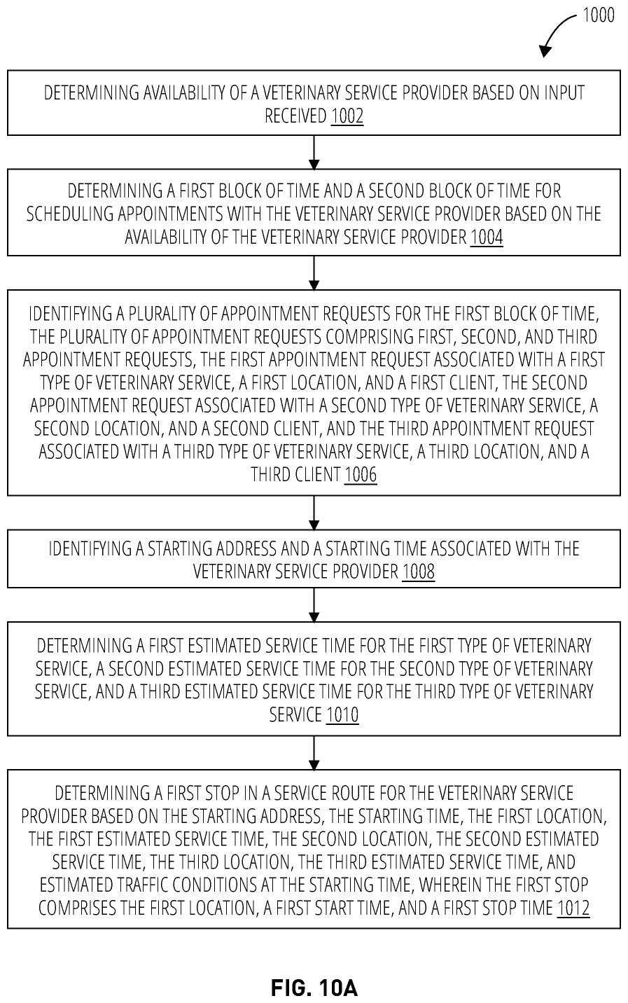

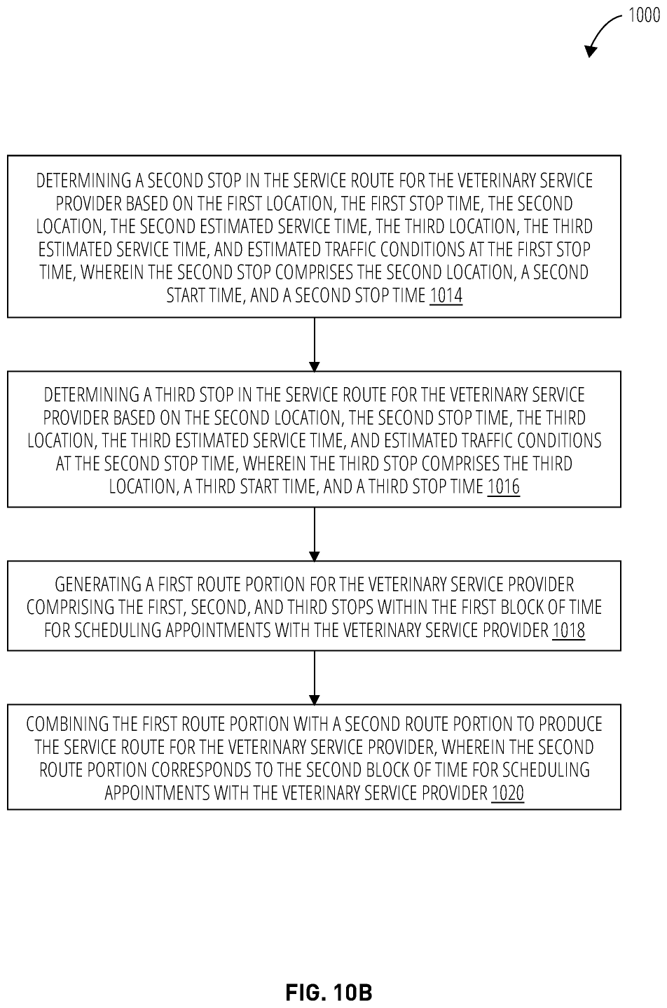

FIGS. 10A and 10B illustrate one embodiment of a logic flow 1000, which may be representative of operations that may be executed in various embodiments in conjunction with techniques disclosed hereby. The logic flow 1000 may be representative of some or all of the operations that may be executed by one or more components/devices/environments described hereby, such as schedule administrator 302, availability manager 402, and/or dynamic route planner 416. The embodiments are not limited in this context.

In the illustrated embodiment, logic flow 1000 may begin at block 1002. At block 1002 “determining availability of a veterinary service provider based on input received” availability of a veterinary service provider may be determined based on input received. For example, availability data 414 may be determined for the veterinary service provider by availability manager 402 based on one or more of appointment data 404, zone data 406, client data 408, provider data 412, and animal data 410. In such examples, the data may be received as input, such as via I/O 308.

Proceeding to block 1004 “determining a first block of time and a second block of time for scheduling appointments with the veterinary service provider based on the availability of the veterinary service provider” first and second blocks of time for scheduling appointments with the veterinary service provider may be determined based on the availability of the veterinary service provider. For example, availability data 414 generated by availability manager 304 may include blocks of time for scheduling appointments with the veterinary service provider determined based on provider data 412.

Continuing to block 1006 “identifying a plurality of appointment requests for the first block of time, the plurality of appointment requests comprising first, second, and third appointment requests, the first appointment request associated with a first type of veterinary service, a first location, and a first client, the second appointment request associated with a second type of veterinary service, a second location, and a second client, and the third appointment request associated with a third type of veterinary service, a third location, and a third client” a plurality of appointment requests for the first block of time may be identified. Further, the plurality of appointment requests may include first, second, and third appointment requests. The first appointment request may be associated with a first type of veterinary service, a first location, and a first client. The second appointment request may be associated with a second type of veterinary service, a second location, and a second client. The third appointment request may be associated with a third type of veterinary service, a third location, and a third client. For example, schedule administrator 302 may receive the plurality of appointment requests via I/O 308 as appointment data 202.

At block 1008 “identifying a starting address and a starting time associated with the veterinary service provider” a starting address and a starting time associated with the veterinary service provider may be identified. For example, schedule administrator 104 may identify a starting address and a starting time associated with the veterinary service provider based on provider data 114 of planning data 102. Proceeding to block 1010 “determining a first estimated service time for the first type of veterinary service, a second estimated service time for the second type of veterinary service, and a third estimated service time for the third type of veterinary service” a first estimated service time may be determined for the first type of veterinary service (i.e., a first veterinary service type), a second estimated service time may be determined for the second type of veterinary service (i.e., a second veterinary service type), and a third estimated service time may be determined for the third type of veterinary service (i.e., a third veterinary service type). For example, duration estimator 432 may generate an estimated service time for each type of veterinary service, such as based on one or more portions of historical data 246.

Continuing to block 1012 “determining a first stop in a service route for the veterinary service provider based on the starting address, the starting time, the first location, the first estimated service time, the second location, the second estimated service time, the third location, the third estimated service time, and estimated traffic conditions at the starting time, wherein the first stop comprises the first location, a first start time, and a first stop time” a first stop comprising a first location, a first start time, and a first stop time in a service route for the veterinary service provider may be determined based on the starting address, the starting time, the first location, the first estimated service time, the second location, the second estimated service time, the third location, the third estimated service time, and estimated traffic conditions at the starting time. For example, the starting address may include a starting location for the veterinary service provider, the first location may include the address of a first appointment, the first start time may include the target start time for the first appointment, and the first stop time may include the target finish time for the first appointment. In many embodiments, dynamic route planner 416 may determine the first stop in the service route for the veterinary service provider. In many such embodiments, dynamic route planner 416 may access current and/or estimated future traffic conditions in determining the first stop. In one embodiment, dynamic route planner 416 may utilize an artificial intelligence algorithm trained based on historical data 246 to determine the first stop.

Proceeding to block 1014 “determining a second stop in the service route for the veterinary service provider based on the first location, the first stop time, the second location, the second estimated service time, the third location, the third estimated service time, and estimated traffic conditions at the first stop time, wherein the second stop comprises the second location, a second start time, and a second stop time” a second stop comprising a second location, a second start time, and a second stop time in the service route for the veterinary service provider may be determined based on the first location, the first stop time, the second location, the second estimated service time, the third location, the third estimated service time, and estimated traffic conditions at the first stop time. For example, the second location may include the address of a second appointment, the second start time may include the target start time for the second appointment, and the second stop time may include the target finish time for the second appointment. In many embodiments, dynamic route planner 416 may determine the second stop in the service route for the veterinary service provider. In many such embodiments, dynamic route planner 416 may access current and/or estimated future traffic conditions in determining the second stop. In one embodiment, dynamic route planner 416 may utilize an artificial intelligence algorithm trained based on historical data 246 to determine the second stop.

At block 1016 “determining a third stop in the service route for the veterinary service provider based on the second location, the second stop time, the third location, the third estimated service time, and estimated traffic conditions at the second stop time, wherein the third stop comprises the third location, a third start time, and a third stop time” a third stop comprising a second location, a second start time, and a second stop time in the service route for the veterinary service provider may be determined based on the second location, the second stop time, the third location, the third estimated service time, and estimated traffic conditions at the second stop time. For example, the third location may include the address of a third appointment, the third start time may include the target start time for the third appointment, and the third stop time may include the target finish time for the third appointment. In many embodiments, dynamic route planner 416 may determine the third stop in the service route for the veterinary service provider. In many such embodiments, dynamic route planner 416 may access current and/or estimated future traffic conditions in determining the third stop. In one embodiment, dynamic route planner 416 may utilize an artificial intelligence algorithm trained based on historical data 246 to determine the third stop.

Continuing to block 1018 “generating a first route portion for the veterinary service provider comprising the first, second, and third stops within the first block of time for scheduling appointments with the veterinary service provider” a first route portion for the veterinary service provider may be generated. The first route portion may include the first, second, and third stops within the first block of time for scheduling appointments with the veterinary service provider. For example, dynamic route planner 416 may generate the first route portion including first, second, and third stops within a morning block of time based on one or more of estimated service time 418, appointment data 420, address data 422, provider data 426, and availability data 424. At block 1020 “combining the first route portion with a second route portion to produce the service route for the veterinary service provider, wherein the second route portion corresponds to the second block of time for scheduling appointments with the veterinary service provider” the first route portion may be combined with a second route portion corresponding to the second block of time for scheduling appointments with the veterinary service provider to produce the service route for the veterinary service provider. For example, dynamic route planner 416 may combine the first and second route portions to produce a service route for the veterinary service provider in provider routes 428.

Some embodiments include notifying the first client of the first start time, the second client of the second start time, and the third client of the third start time. For example, schedule administrator 302 may utilize I/O 308 to notify clients about corresponding start times. In some such examples, schedule administrator 302 may also utilize I/O 308 to notify clients about corresponding stop times. Various embodiments include notifying the veterinary service provider of the route. For example, schedule administrator 302 may utilize I/O 308 to notify providers of provider routes 316. Various embodiments include identifying first medical data regarding the first stop, second medical data regarding the second stop, and third medical data regarding the third stop; and notifying the veterinary service provider of the first medical data, the second medical data, and the third medical data. For example, the first, second, and third medical data may be included in client information 318. In one embodiment, notification of the first, second, and third medical data to the veterinary service provider may proceed as follows: notify the veterinary service provider of the first medical data between the starting time associated with the veterinary service provider and the first start time; notify the veterinary service provider of the second medical data between the first stop time and the second start time; and notify the veterinary service provider of the third medical data between the second stop time and the third start time. Many embodiments include identifying a maximum number of appointments for the first block of time and rejecting a fourth appointment request for the first block of time based on the maximum number of appointments for the first block of time. Some embodiments include adding a break period between the first route portion and the second route portion to produce the service route for the veterinary service provider.

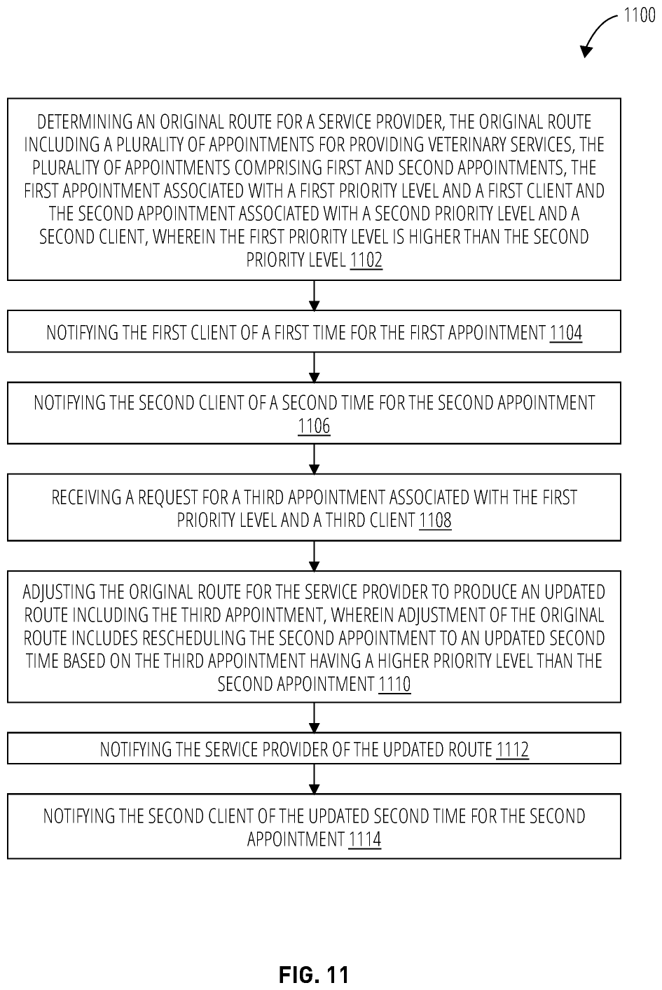

FIG. 11 illustrates one embodiment of a logic flow 1100, which may be representative of operations that may be executed in various embodiments in conjunction with techniques disclosed hereby. The logic flow 1100 may be representative of some or all of the operations that may be executed by one or more components/devices/environments described hereby, such as schedule administrator 302 and/or dynamic route planner 416. The embodiments are not limited in this context.

In the illustrated embodiment, logic flow 1100 may begin at block 1102. At block 1102 “determining an original route for a service provider, the original route including a plurality of appointments for providing veterinary services, the plurality of appointments comprising first and second appointments, the first appointment associated with a first priority level and a first client and the second appointment associated with a second priority level and a second client, wherein the first priority level is higher than the second priority level” an original route may be determined for a service provider. The original route may include a plurality of appointments for providing veterinary services. The plurality of appointments may include first and second appointments. The first appointment associated with a first priority level and a first client, and the second appointment associated with a second priority level and a second client, wherein the first priority level is higher than the second priority level. For example, dynamic route planner 416 may generate an original provider route within provider routes 428 having a first appointment associated with a first priority level and a first client and a second appointment associated with a second priority level and a second client.

At block 1104 “notifying the first client of a first time for the first appointment” the first client may be notified of the first time for the first appointment. For example, schedule administrator 302 may utilize I/O 308 to notify a first client of the start time for the first client's appointment in schedule 322. Continuing to block 1106 “notifying the second client of a second time for the second appointment” the second client may be notified of the second time for the second appointment. For example, schedule administrator 302 may utilize I/O 308 to notify a second client of the start time for the second client's appointment in schedule 322.

Proceeding to block 1108 “receiving a request for a third appointment associated with the first priority level and a third client” a request for a third appointment associated with the first priority level and a third client may be received. For example, a request for a third appointment associated with the first priority level and a third client may be received by schedule administrator 302 via I/O 308. At block 1110 “adjusting the original route for the service provider to produce an updated route including the third appointment, wherein adjustment of the original route includes rescheduling the second appointment to an updated second time based on the third appointment having a higher priority level than the second appointment” the original route for the service provider may be adjusted to produce an updated route including the third appointment. Adjustment of the original route may include rescheduling the second appointment to an updated second time based on the third appointment having a higher priority level than the second appointment. For example, dynamic route planner 310 may produce an updated route that reschedules the second appointment to an updated second time based on the third appointment having a higher priority level than the second appointment.

At block 1112 “notifying the service provider of the updated route” the service provider may be notified of the updated route. For example, schedule administrator 302 may utilize I/O 308 to provide updates 324 comprising the updated route to the service provider. Continuing to block 1114 “notifying the second client of the updated second time for the second appointment” the second client may be notified of the updated second time for the second appointment. For example, schedule administrator 302 may utilize I/O 308 to provide updates 324 comprising the updated second time for the second appointment to the second client.

In various embodiments, the third appointment is associated with a second service zone located outside of the first service zone. In various such embodiments, the third appointment is added to the updated route in response to unavailability of another service provider in the second service zone. In some embodiments, the plurality of appointments included in the original route includes a fourth appointment associated with a fourth client. Some such embodiments include notifying the fourth client of an original time window for the fourth appointment and notifying the fourth client of an updated time window for the fourth appointment in response to production of the updated route. In various such embodiments, the updated time window is a subset of the original time window. For example, the original time window may include between 8 am and 12 pm and the updated time window may include 11 am to 12p. Many embodiments include providing the plurality of appointments to an artificial intelligence algorithm trained on historical data comprising a plurality of historical appointments to determine the original route. In many such embodiments, at least one of the plurality of historical appointments are associated with a corresponding location, and a corresponding service time. For example, dynamic route planner 416 may utilize an artificial intelligence algorithm trained on historical data 430.

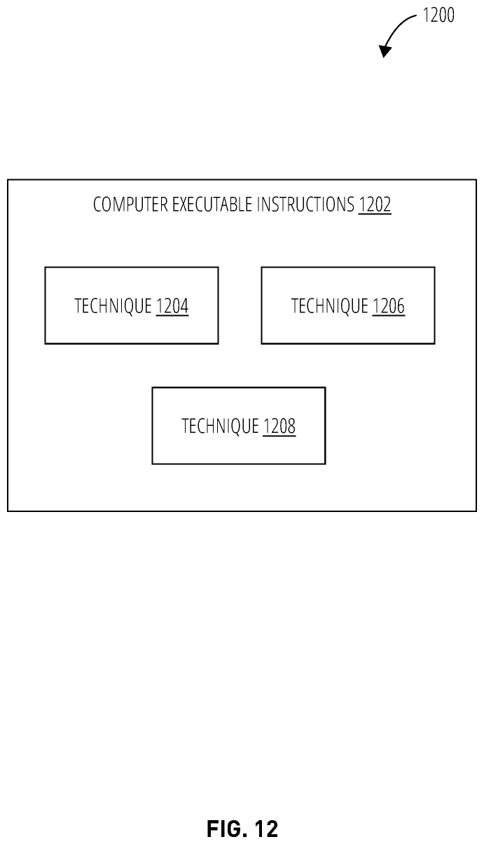

FIG. 12 illustrates computer-readable storage medium 1200. Computer-readable storage medium 1200 may comprise any non-transitory computer-readable storage medium or machine-readable storage medium, such as an optical, magnetic or semiconductor storage medium. In various embodiments, computer-readable storage medium 1200 may comprise an article of manufacture. In some embodiments, computer-readable storage medium 1200 may store computer executable instructions 1202 with which circuitry (e.g., processor 502, processor 602, processor 706, etc.) can execute. For example, computer executable instructions 1202 can include instructions to implement operations described hereby, such as with respect to technique 1204, technique 1206, and/or technique 1208. In various embodiments, technique 1204 may refer to the logic flow 900 of FIG. 9, technique 1206 may refer to the logic flow 1000 of FIG. 10A, and technique 1208 may refer to the logic flow 1100 of FIG. 11. Examples of computer-readable storage medium 1200 or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions 1202 may include any suitable type of code, such as source code, compiled code, interpreted code, executable code, static code, dynamic code, object-oriented code, visual code, and the like.

FIG. 13 illustrates a diagrammatic representation of a machine 1300 in the form of a computer system within which a set of instructions may be executed for causing the machine to perform any one or more of the methodologies discussed herein. More specifically, FIG. 13 shows a diagrammatic representation of the machine 1300 in the example form of a computer system, within which instructions 1308 (e.g., software, a program, an application, an applet, an app, or other executable code) for causing the machine 1300 to perform any one or more of the methodologies discussed herein may be executed. For example, the instructions 1308 may cause the machine 1300 to execute one or more of the techniques 1204, 1206, 1208 of FIG. 12. More generally, the instructions 1308 may cause the machine 1300 to facilitate one or more embodiments disclosed hereby.

The instructions 1308 transform the general, non-programmed machine 1300 into a particular machine 1300 programmed to carry out the described and illustrated functions in a specific manner. In alternative embodiments, the machine 1300 operates as a standalone device or may be coupled (e.g., networked) to other machines. In a networked deployment, the machine 1300 may operate in the capacity of a server machine or a client machine in a server-client network environment, or as a peer machine in a peer-to-peer (or distributed) network environment. The machine 1300 may comprise, but not be limited to, a server computer, a client computer, a personal computer (PC), a tablet computer, a laptop computer, a netbook, a set-top box (STB), a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 1308, sequentially or otherwise, that specify actions to be taken by the machine 1300. Further, while only a single machine 1300 is illustrated, the term “machine” shall also be taken to include a collection of machines 200 that individually or jointly execute the instructions 1308 to perform any one or more of the methodologies discussed herein.

The machine 1300 may include processors 1302, memory 1304, and I/O components 1342, which may be configured to communicate with each other such as via a bus 1344. In an example embodiment, the processors 1302 (e.g., a Central Processing Unit (CPU), a Reduced Instruction Set Computing (RISC) processor, a Complex Instruction Set Computing (CISC) processor, a Graphics Processing Unit (GPU), a Digital Signal Processor (DSP), an ASIC, a Radio-Frequency Integrated Circuit (RFIC), another processor, or any suitable combination thereof) may include, for example, a processor 1306 and a processor 1310 that may execute the instructions 1308. The term “processor” is intended to include multi-core processors that may comprise two or more independent processors (sometimes referred to as “cores”) that may execute instructions contemporaneously. Although FIG. 13 shows multiple processors 1302, the machine 1300 may include a single processor with a single core, a single processor with multiple cores (e.g., a multi-core processor), multiple processors with a single core, multiple processors with multiples cores, or any combination thereof.

The memory 1304 may include a main memory 1312, a static memory 1314, and a storage unit 1316, both accessible to the processors 1302 such as via the bus 1344. The main memory 1304, the static memory 1314, and storage unit 1316 store the instructions 1308 embodying any one or more of the methodologies or functions described herein. The instructions 1308 may also reside, completely or partially, within the main memory 1312, within the static memory 1314, within machine-readable medium 1318 within the storage unit 1316, within at least one of the processors 1302 (e.g., within the processor's cache memory), or any suitable combination thereof, during execution thereof by the machine 1300.

The I/O components 1342 may include a wide variety of components to receive input, provide output, produce output, transmit information, exchange information, capture measurements, and so on. The specific I/O components 1342 that are included in a particular machine will depend on the type of machine. For example, portable machines such as mobile phones will likely include a touch input device or other such input mechanisms, while a headless server machine will likely not include such a touch input device. It will be appreciated that the I/O components 1342 may include many other components that are not shown in FIG. 13. The I/O components 1342 are grouped according to functionality merely for simplifying the following discussion and the grouping is in no way limiting. In various example embodiments, the I/O components 1342 may include output components 1328 and input components 1330. The output components 1328 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 1330 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), and the like.

In further example embodiments, the I/O components 1342 may include biometric components 1332, motion components 1334, environmental components 1336, or position components 1338, among a wide array of other components. For example, the biometric components 1332 may include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram-based identification), and the like. The motion components 1334 may include acceleration sensor components (e.g., accelerometer), gravitation sensor components, rotation sensor components (e.g., gyroscope), and so forth. The environmental components 1336 may include, for example, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 1338 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 1342 may include communication components 1340 operable to couple the machine 1300 to a network 1320 or devices 1322 via a coupling 1324 and a coupling 1326, respectively. For example, the communication components 1340 may include a network interface component or another suitable device to interface with the network 1320. In further examples, the communication components 1340 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 1322 may be another machine or any of a wide variety of peripheral devices (e.g., a peripheral device coupled via a USB).

Moreover, the communication components 1340 may detect identifiers or include components operable to detect identifiers. For example, the communication components 1340 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 1340, such as location via Internet Protocol (IP) geolocation, location via Wi-Fi® signal triangulation, location via detecting an NFC beacon signal that may indicate a particular location, and so forth.

The various memories (i.e., memory 1304, main memory 1312, static memory 1314, and/or memory of the processors 1302) and/or storage unit 1316 may store one or more sets of instructions and data structures (e.g., software) embodying or utilized by any one or more of the methodologies or functions described herein. These instructions (e.g., the instructions 1308), when executed by processors 1302, cause various operations to implement the disclosed embodiments.

As used herein, the terms “machine-storage medium,” “device-storage medium,” “computer-storage medium” mean the same thing and may be used interchangeably in this disclosure. The terms refer to a single or multiple storage devices and/or media (e.g., a centralized or distributed database, and/or associated caches and servers) that store executable instructions and/or data. The terms shall accordingly be taken to include, but not be limited to, solid-state memories, and optical and magnetic media, including memory internal or external to processors. Specific examples of machine-storage media, computer-storage media and/or device-storage media include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks. The terms “machine-storage media,” “computer-storage media,” and “device-storage media” specifically exclude carrier waves, modulated data signals, and other such media, at least some of which are covered under the term “signal medium” discussed below.

In various example embodiments, one or more portions of the network 1320 may be an ad hoc network, an intranet, an extranet, a VPN, a LAN, a WLAN, a WAN, a WWAN, a MAN, the Internet, a portion of the Internet, a portion of the PSTN, a plain old telephone service (POTS) network, a cellular telephone network, a wireless network, a Wi-Fi® network, another type of network, or a combination of two or more such networks. For example, the network 1320 or a portion of the network 1320 may include a wireless or cellular network, and the coupling 1324 may be a Code Division Multiple Access (CDMA) connection, a Global System for Mobile communications (GSM) connection, or another type of cellular or wireless coupling. In this example, the coupling 1324 may implement any of a variety of types of data transfer technology, such as Single Carrier Radio Transmission Technology (1×RTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard-setting organizations, other long range protocols, or other data transfer technology.

The instructions 1308 may be transmitted or received over the network 1320 using a transmission medium via a network interface device (e.g., a network interface component included in the communication components 1340) and utilizing any one of a number of well-known transfer protocols (e.g., hypertext transfer protocol (HTTP)). Similarly, the instructions 1308 may be transmitted or received using a transmission medium via the coupling 1326 (e.g., a peer-to-peer coupling) to the devices 1322. The terms “transmission medium” and “signal medium” mean the same thing and may be used interchangeably in this disclosure. The terms “transmission medium” and “signal medium” shall be taken to include any intangible medium that is capable of storing, encoding, or carrying the instructions 1308 for execution by the machine 1300, and includes digital or analog communications signals or other intangible media to facilitate communication of such software. Hence, the terms “transmission medium” and “signal medium” shall be taken to include any form of modulated data signal, carrier wave, and so forth. The term “modulated data signal” means a signal that has one or more of its characteristics set or changed in such a matter as to encode information in the signal.

There are a number of example embodiments described herein.

Example 1 is a computer-implemented method, comprising receiving a request for an appointment, the request including appointment information comprising a veterinary service type, a client, an animal, and a service location; identifying client data associated with the client and animal data associated with the animal, wherein the animal data includes an animal type; determining a location type based on at least one of the animal data, the client data, and the appointment information; generating an estimated service time for the appointment by providing two or more of the veterinary service type, the animal type, and the location type to an artificial intelligence algorithm trained based on historical data comprising a plurality of historical appointments; and scheduling the appointment based on the estimated service time and the service location.

Example 2 is the computer-implemented method of Example 1 that may optionally include generating a route for a veterinary service provider based on the estimated service time and the service location, wherein the route includes the appointment and at least one additional appointment.

Example 3 is the computer-implemented method of Example 1 that may optionally include that at least one of the plurality of historical appointments are associated with two or more of a corresponding service time, a corresponding veterinary service type, a corresponding animal type, and a corresponding location type.

Example 4 is the computer-implemented method of Example 1 that may optionally include determining a service provider for the appointment; and generating the estimated service time for the appointment by providing the service provider and the two or more of the veterinary service type, the animal type, and the location type to the artificial intelligence algorithm.

Example 5 is the computer-implemented method of Example 4 that may optionally include that at least one of the plurality of historical appointments are associated with a corresponding service provider and two or more of a corresponding service time, a corresponding veterinary service type, a corresponding animal type, and a corresponding location type.

Example 6 is the computer-implemented method of Example 1 that may optionally include generating the estimated service time for the appointment by providing the client data and the two or more of the veterinary service type, the animal type, and the location type to the artificial intelligence algorithm.

Example 7 is the computer-implemented method of Example 1 that may optionally include that the animal type is provided to the artificial intelligence algorithm in animal data including the animal type and data indicative of at least one historical interaction with the animal.

Example 8 is the computer-implemented method of Example 1 that may optionally include generating a multi-dimensional vector for the appointment, wherein the multi-dimensional vector includes a first value generated based on the veterinary service type, a second value generated based on the animal type, and a third value generated based on the location type; and providing the multi-dimensional vector to the artificial intelligence algorithm to provide the two or more of the veterinary service type, the animal type, and the location type to the artificial intelligence algorithm for generation of the estimated service time.

Example 9 is the computer-implemented method of Example 1 that may optionally include determining zone data based on the service location, the zone data comprising a service zone corresponding to the service location and a legal zone corresponding to the service location; and determining a service provider for the appointment based on the zone data.

Example 10 is an apparatus comprising one or more processors configured to perform the computer-implemented method of any of Examples 1 to 9.

Example 11 is a non-transitory machine-readable medium having executable instructions to cause one or more processing units to perform the computer-implemented method of any of Examples 1 to 9.

Example 12 is a computer-implemented method, comprising determining availability of a veterinary service provider based on input received; determining a first block of time and a second block of time for scheduling appointments with the veterinary service provider based on the availability of the veterinary service provider; identifying a plurality of appointment requests for the first block of time, the plurality of appointment requests comprising first, second, and third appointment requests, the first appointment request associated with a first type of veterinary service, a first location, and a first client, the second appointment request associated with a second type of veterinary service, a second location, and a second client, and the third appointment request associated with a third type of veterinary service, a third location, and a third client; identifying a starting address and a starting time associated with the veterinary service provider; determining a first estimated service time for the first type of veterinary service, a second estimated service time for the second type of veterinary service, and a third estimated service time for the third type of veterinary service; determining a first stop in a service route for the veterinary service provider based on the starting address, the starting time, the first location, the first estimated service time, the second location, the second estimated service time, the third location, the third estimated service time, and estimated traffic conditions at the starting time, wherein the first stop comprises the first location, a first start time, and a first stop time; determining a second stop in the service route for the veterinary service provider based on the first location, the first stop time, the second location, the second estimated service time, the third location, the third estimated service time, and estimated traffic conditions at the first stop time, wherein the second stop comprises the second location, a second start time, and a second stop time; determining a third stop in the service route for the veterinary service provider based on the second location, the second stop time, the third location, the third estimated service time, and estimated traffic conditions at the second stop time, wherein the third stop comprises the third location, a third start time, and a third stop time; generating a first route portion for the veterinary service provider comprising the first, second, and third stops within the first block of time for scheduling appointments with the veterinary service provider; and combining the first route portion with a second route portion to produce the service route for the veterinary service provider, wherein the second route portion corresponds to the second block of time for scheduling appointments with the veterinary service provider.

Example 13 is the computer-implemented method of Example 12 that may optionally include notifying the first client of the first start time, the second client of the second start time, and the third client of the third start time.

Example 14 is the computer-implemented method of Example 12 that may optionally include notifying the veterinary service provider of the route.

Example 15 is the computer-implemented method of Example 12 that may optionally include identifying first medical data regarding the first stop, second medical data regarding the second stop, and third medical data regarding the third stop; and notifying the veterinary service provider of the first medical data, the second medical data, and the third medical data.

Example 16 is the computer-implemented method of Example 15 that may optionally include notifying the veterinary service provider of the first medical data between the starting time associated with the veterinary service provider and the first start time; notifying the veterinary service provider of the second medical data between the first stop time and the second start time; and notifying the veterinary service provider of the third medical data between the second stop time and the third start time.

Example 17 is the computer-implemented method of Example 12 that may optionally include identifying a maximum number of appointments for the first block of time; and rejecting a fourth appointment request for the first block of time based on the maximum number of appointments for the first block of time.

Example 18 is the computer-implemented method of Example 12 that may optionally include adding a break period between the first route portion and the second route portion to produce the service route for the veterinary service provider.

Example 19 is an apparatus comprising one or more processors configured to perform the computer-implemented method of any of Examples 12 to 18.

Example 20 is a non-transitory machine-readable medium having executable instructions to cause one or more processing units to perform the computer-implemented method of any of Examples 12 to 18.

Example 21 is a computer-implemented method, comprising determining an original route for a service provider, the original route including a plurality of appointments for providing veterinary services, the plurality of appointments comprising first and second appointments, the first appointment associated with a first priority level and a first client, and the second appointment associated with a second priority level and a second client, wherein the first priority level is higher than the second priority level; notifying the first client of a first time for the first appointment; notifying the second client of a second time for the second appointment; receiving a request for a third appointment associated with the first priority level and a third client; adjusting the original route for the service provider to produce an updated route including the third appointment, wherein adjustment of the original route includes rescheduling the second appointment to an updated second time based on the third appointment having a higher priority level than the second appointment; notifying the service provider of the updated route; and notifying the second client of the updated second time for the second appointment.

Example 22 is the computer-implemented method of Example 21 that may optionally include that the third appointment is associated with a second service zone located outside of the first service zone.

Example 23 is the computer-implemented method of Example 22 that may optionally include that the third appointment is added to the updated route in response to unavailability of another service provider in the second service zone.

Example 24 is the computer-implemented method of Example 21 that may optionally include that the plurality of appointments included in the original route includes a fourth appointment associated with a fourth client, and comprising notifying the fourth client of an original time window for the fourth appointment; and notifying the fourth client of an updated time window for the fourth appointment in response to production of the updated route.

Example 25 is the computer-implemented method of Example 24 that may optionally include that the updated time window is a subset of the original time window.

Example 26 is the computer-implemented method of Example 21 that may optionally include providing the plurality of appointments to an artificial intelligence algorithm trained on historical data comprising a plurality of historical appointments to determine the original route.

Example 27 is the computer-implemented method of Example 26 that may optionally include that at least one of the plurality of historical appointments are associated with a corresponding location, and a corresponding service time.

Example 28 is an apparatus comprising one or more processors configured to perform the computer-implemented method of any of Examples 21 to 27.

Example 29 is a non-transitory machine-readable medium having executable instructions to cause one or more processing units to perform the computer-implemented method of any of Examples 21 to 27.

Terms used herein should be accorded their ordinary meaning in the relevant arts, or the meaning indicated by their use in context, but if an express definition is provided, that meaning controls.

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to a single one or multiple ones. Additionally, the words “herein,” “above,” “below” and words of similar import, when used in this application, refer to this application as a whole and not to any particular portions of this application. When the claims use the word “or” in reference to a list of two or more items, that word covers all of the following interpretations of the word: any of the items in the list, all of the items in the list and any combination of the items in the list, unless expressly limited to one or the other. Any terms not expressly defined herein have their conventional meaning as commonly understood by those having skill in the relevant art(s).

What is claimed is:

1. A computer-implemented method, comprising:
receiving a request for an appointment, the request including appointment information comprising a veterinary service type, a client, an animal, and a service location;
identifying client data associated with the client and animal data associated with the animal, wherein the animal data includes an animal type;
determining a location type based on at least one of the animal data, the client data, and the appointment information;
generating an estimated service time for the appointment by providing two or more of the veterinary service type, the animal type, and the location type to an artificial intelligence algorithm trained based on historical data comprising a plurality of historical appointments; and
scheduling the appointment based on the estimated service time and the service location.

2. The computer-implemented method of claim 1, comprising generating a route for a veterinary service provider based on the estimated service time and the service location, wherein the route includes the appointment and at least one additional appointment.

3. The computer-implemented method of claim 1, wherein at least one of the plurality of historical appointments are associated with two or more of a corresponding service time, a corresponding veterinary service type, a corresponding animal type, and a corresponding location type.

4. The computer-implemented method of claim 1, comprising:
determining a service provider for the appointment; and
generating the estimated service time for the appointment by providing the service provider and the two or more of the veterinary service type, the animal type, and the location type to the artificial intelligence algorithm.

5. The computer-implemented method of claim 4, wherein at least one of the plurality of historical appointments are associated with a corresponding service provider and two or more of a corresponding service time, a corresponding veterinary service type, a corresponding animal type, and a corresponding location type.

6. The computer-implemented method of claim 1, comprising generating the estimated service time for the appointment by providing the client data and the two or more of the veterinary service type, the animal type, and the location type to the artificial intelligence algorithm.

7. The computer-implemented method of claim 1, wherein the animal type is provided to the artificial intelligence algorithm in animal data including the animal type and data indicative of at least one historical interaction with the animal.

8. The computer-implemented method of claim 1, comprising:
generating a multi-dimensional vector for the appointment, wherein the multi-dimensional vector includes a first value generated based on the veterinary service type, a second value generated based on the animal type, and a third value generated based on the location type;
providing the multi-dimensional vector to the artificial intelligence algorithm to provide the two or more of the veterinary service type, the animal type, and the location type to the artificial intelligence algorithm for generation of the estimated service time.

9. The computer-implemented method of claim 1, comprising:
determining zone data based on the service location, the zone data comprising a service zone corresponding to the service location and a legal zone corresponding to the service location; and
determining a service provider for the appointment based on the zone data.

10. An apparatus, the apparatus comprising:
a processor; and
memory comprising instructions that when executed by the processor cause the processor to:
receive a request for an appointment, the request including appointment information comprising a veterinary service type, a client, an animal, and a service location;
identify client data associated with the client and animal data associated with the animal, wherein the animal data includes an animal type;
determine a location type based on at least one of the animal data, the client data, and the appointment information;
generate an estimated service time for the appointment by providing two or more of the veterinary service type, the animal type, and the location type to an artificial intelligence algorithm trained based on historical data comprising a plurality of historical appointments; and
schedule the appointment based on the estimated service time and the service location.

11. The apparatus of claim 10, wherein the instructions, when executed by the processor, further cause the processor to generate a route for a veterinary service provider based on the estimated service time and the service location, wherein the route includes the appointment and at least one additional appointment.

12. The apparatus of claim 10, wherein at least one of the plurality of historical appointments are associated with two or more of a corresponding service time, a corresponding veterinary service type, a corresponding animal type, and a corresponding location type.

13. The apparatus of claim 10, wherein the instructions, when executed by the processor, further cause the processor to:
determine a service provider for the appointment; and
generate the estimated service time for the appointment by providing the service provider and the two or more of the veterinary service type, the animal type, and the location type to the artificial intelligence algorithm.

14. The apparatus of claim 13, wherein at least one of the plurality of historical appointments are associated with a corresponding service provider and two or more of a corresponding service time, a corresponding veterinary service type, a corresponding animal type, and a corresponding location type.

15. The apparatus of claim 10 wherein the animal type is provided to the artificial intelligence algorithm in animal data including the animal type and data indicative of at least one historical interaction with the animal.

16. At least one non-transitory computer-readable medium comprising a set of instructions that, in response to being executed by a processor circuit, cause the processor circuit to:
receive a request for an appointment, the request including appointment information comprising a veterinary service type, a client, an animal, and a service location;
identify client data associated with the client and animal data associated with the animal, wherein the animal data includes an animal type;
determine a location type based on at least one of the animal data, the client data, and the appointment information;
generate an estimated service time for the appointment by providing two or more of the veterinary service type, the animal type, and the location type to an artificial intelligence algorithm trained based on historical data comprising a plurality of historical appointments; and
schedule the appointment based on the estimated service time and the service location.

17. The at least one non-transitory computer-readable medium of claim 16, wherein the set of instructions, in response to execution by the processor circuit, further cause the processor circuit to:
determine a service provider for the appointment; and
generate the estimated service time for the appointment by providing the service provider and the two or more of the veterinary service type, the animal type, and the location type to the artificial intelligence algorithm.

18. The at least one non-transitory computer-readable medium of claim 16, wherein the set of instructions, in response to execution by the processor circuit, further cause the processor circuit to generate the estimated service time for the appointment by providing the client data and the two or more of the veterinary service type, the animal type, and the location type to the artificial intelligence algorithm.

19. The at least one non-transitory computer-readable medium of claim 1, wherein the set of instructions, in response to execution by the processor circuit, further cause the processor circuit to:
generate a multi-dimensional vector for the appointment, wherein the multi-dimensional vector includes a first value generated based on the veterinary service type, a second value generated based on the animal type, and a third value generated based on the location type;
provide the multi-dimensional vector to the artificial intelligence algorithm to provide the two or more of the veterinary service type, the animal type, and the location type to the artificial intelligence algorithm for generation of the estimated service time.

20. The at least one non-transitory computer-readable medium of claim 1, wherein the set of instructions, in response to execution by the processor circuit, further cause the processor circuit to:
determine zone data based on the service location, the zone data comprising a service zone corresponding to the service location and a legal zone corresponding to the service location; and
determine a service provider for the appointment based on the zone data.
